(19) United States
(12) Patent Application Publication
(10) Pub. No.: US 2023/0279121 A1
(43) Pub. Date: Sep. 7, 2023

(54) ANTIBODIES TARGETING EGFR AND USE THEREOF

(71) Applicant: Dragonfly Therapeutics, Inc., Waltham, MA (US)

(72) Inventors: Asya Grinberg, Lexington, MA (US); Zong Sean Juo, Taipei (TW); Katia Liharska, Billerica, MA (US); Christopher Ryan Morgan, Southborough, MA (US)

(21) Appl. No.: 18/040,501

(22) PCT Filed: Aug. 5, 2021
(86) PCT No.: PCT/US2021/044688

§ 371 Date: Feb. 3, 2023

Related U.S. Application Data
(60) Provisional application No. 63/061,507, filed on Aug. 5, 2020.

(51) Int. Cl. C07K 16/28; C12N 15/63; A61P 35/00

(52) U.S. Cl. CPC C07K 16/2863; C12N 15/63; A61P 35/00; C07K 2317/565; C07K 2317/92

(57) ABSTRACT

Disclosed are proteins with antibody heavy chain and light chain variable domains that can be paired to form an antigen-binding site targeting EGFR on a cell, pharmaceutical compositions comprising such proteins, and therapeutic methods using such proteins and pharmaceutical compositions, including for the treatment of cancer.

### CROSS-REFERENCE TO RELATED APPLICATIONS

This application claims the benefit of and priority to U.S. Provisional Patent Application No. 63/061,507, filed Aug. 5, 2020, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Aug. 4, 2021, is named DFY-083WO_SL.txt and is 61,970 bytes in size.

### FIELD OF THE INVENTION

### Background

Cancer continues to be a significant health problem despite the substantial research efforts and scientific advances reported in the literature for treating this disease. Some of the most frequently diagnosed cancers in adults include prostate cancer, breast cancer, and lung cancer. Hematological malignancies, though less frequent than solid cancers, have low survival rates. Current treatment options for these cancers are not effective for all patients and/or can have substantial adverse side effects. Other types of cancer also remain challenging to treat using existing therapeutic options.

The epidermal growth factor receptor (EGFR; ErbB-1; HER1) is a transmembrane protein that is a receptor for members of the epidermal growth factor family (EGF family) of extracellular protein ligands. Upon binding of its specific ligands, including epidermal growth factor and transforming growth factor α (TGFα), EGFR undergoes a transition from an inactive monomeric form to an active homodimer or heterodimer with other ErbB family receptors. The dimerization stimulates its intrinsic intracellular protein-tyrosine kinase activity, and elicits downstream signaling cascades, leading to DNA synthesis and cell proliferation. EGFR is involved in modulation of phenotypes such as cell migration, adhesion, and proliferation.

Mutations that lead to EGFR overexpression or overactivity have been associated with a number of cancers, including non-small cell lung cancer, anal cancers, glioblastoma and epithelial tumors of the head and neck. These somatic mutations involving EGFR lead to its constant activation, which produces uncontrolled cell division. In glioblastoma a more or less specific mutation of EGFR, called EGFRvIII is often observed. Mutations, amplifications or misregulations of EGFR or family members are implicated in other solid tumors, including colorectal cancer, renal cell carcinoma, bladder cancer, cervical cancer, ovarian cancer, pancreatic cancer, and liver cancer.

Anti-EGFR monoclonal antibodies, such as cetuximab, panitumumab, necitumumab, and zalutumumab, have been developed. However, there remains a need for new and useful antibodies and related therapies for use in treatment of cancer that have great efficacy and reduced adverse effects.

### SUMMARY

The present application provides antigen-binding sites that bind human EGFR. These antigen-binding sites bind various epitopes in an extracellular domain of EGFR. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating EGFR-associated diseases such as cancer.

Accordingly, one aspect of the present application provides an antigen-binding site that binds EGFR, comprising: (i) a heavy chain variable domain (VH) comprising complementarity-determining region 1 (CDR1), complementarity-determining region 2 (CDR2), and complementarity-determining region 3 (CDR3) sequences of SEQ ID NOs: 2, 23, and 4, respectively, and a light chain variable domain (VL) comprising CDR1, CDR2, and CDR3 sequences of SEQ ID NOs: 6, 7, and 17, respectively; or (ii) a VH comprising CDR1, CDR2, and CDR3 sequences of SEQ ID NOs: 2, 12, and 4, respectively, and a VL comprising CDR1, CDR2, and CDR3 sequences of SEQ ID NOs: 6, 7, and 8, respectively.

In some embodiments, the VH comprises CDR1, CDR2, and CDR3 sequences of SEQ ID NOs: 2, 23, and 4, respectively; and the VL comprises CDR1, CDR2, and CDR3 sequences of SEQ ID NOs: 6, 7, and 17, respectively. In some embodiments, the VH comprises CDR1, CDR2, and CDR3 sequences of SEQ ID NOs: 2, 12, and 4, respectively; and the VL comprises CDR1, CDR2, and CDR3 sequences of SEQ ID NOs: 6, 7, and 17, respectively. In some embodiments, the VH comprises CDR1, CDR2, and CDR3 sequences of SEQ ID NOs: 2, 3, and 4, respectively; and the VL comprises CDR1, CDR2, and CDR3 sequences of SEQ ID NOs: 6, 7, and 17, respectively. In some embodiments, the VH comprises CDR1, CDR2, and CDR3 sequences of SEQ ID NOs: 2, 12, and 4, respectively; and the VL comprises CDR1, CDR2, and CDR3 sequences of SEQ ID NOs: 6, 7, and 8, respectively.

In some embodiments, the VH comprises an amino acid sequence at least 90% identical to SEQ ID NO:11 and the VL comprises an amino acid sequence at least 90% identical to SEQ ID NO:16. In some embodiments, the VH comprises the amino acid sequence of SEQ ID NO:11 and the VL comprises the amino acid sequence of SEQ ID NO:16. In some embodiments, the VH comprises the amino acid sequence of SEQ ID NO:32 and the VL comprises the amino acid sequence of SEQ ID NO:33.

In some embodiments, the VH comprises an amino acid sequence at least 90% identical to SEQ ID NO:1 and the VL comprises an amino acid sequence at least 90% identical to SEQ ID NO:16. In some embodiments, the VH comprises the amino acid sequence of SEQ ID NO:1 and the VL comprises the amino acid sequence of SEQ ID NO:16.

In some embodiments, the VH comprises an amino acid sequence at least 90% identical to SEQ ID NO:11 and the VL comprises an amino acid sequence at least 90% identical to SEQ ID NO:13. In some embodiments, the VH comprises the amino acid sequence of SEQ ID NO:11 and the VL comprises the amino acid sequence of SEQ ID NO:13.

Another aspect of the present application provides an antigen-binding site comprising a VH comprising an amino acid sequence at least 90% identical to SEQ ID NO:1 and a VL comprising an amino acid sequence at least 90% identical to SEQ ID NO:5, wherein the VH comprises an S62R substitution relative to SEQ ID NO:1 and/or the VL comprises a D92R substitution and/or an F87Y substitution relative to SEQ ID NO:5, numbered under the Chothia numbering scheme.

In some embodiments of any one of the aspects above, the VH comprises an S62R substitution relative to SEQ ID NO:1, numbered under the Chothia numbering scheme. In some embodiments, the VL comprises a D92R substitution relative to SEQ ID NO:5, numbered under the Chothia numbering scheme. In some embodiments, the VH comprises an S62R substitution relative to SEQ ID NO:1 and the VL comprises a D92R substitution relative to SEQ ID NO:5, numbered under the Chothia numbering scheme. In some embodiments, the VL comprises an F87Y substitution relative to SEQ ID NO:5, numbered under the Chothia numbering scheme.

In some embodiments, the antigen-binding site is present as a single-chain fragment variable (scFv), wherein the scFv comprises a sequence selected from SEQ ID NOs: 14, 18, 20, and 24.

In some embodiments, the antigen-binding site binds human EGFR with a dissociation constant (K_{D}) smaller than or equal to 5 nM, as measured by surface plasmon resonance (SPR). In some embodiments, the antigen-binding site binds rhesus macaque EGFR with a dissociation constant (K_{D}) smaller than or equal to 6 nM, as measured by surface plasmon resonance (SPR).

Another aspect of the present application provides a protein comprising an antigen-binding site as disclosed herein. In some embodiments, the protein further comprises an antibody heavy chain constant region. In some embodiments, the antibody heavy chain constant region is a human IgG heavy chain constant region. In some embodiments, the antibody heavy chain constant region is a human IgG1 heavy chain constant region. In some embodiments, each polypeptide chain of the antibody heavy chain constant region comprises an amino acid sequence at least 90% identical to SEQ ID NO:26.

In some embodiments, at least one polypeptide chain of the antibody heavy chain constant region comprises one or more mutations, relative to SEQ ID NO:26, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system. In some embodiments, at least one polypeptide chain of the antibody heavy chain constant region comprises one or more mutations, relative to SEQ ID NO:26, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system.

In some embodiments, one polypeptide chain of the antibody heavy chain constant region comprises one or more mutations, relative to SEQ ID NO:26, at one or more positions selected from Q347, Y349, L351, S354, E356, E357, K360, Q362, S364, T366, L368, K370, K392, T394, D399, S400, D401, F405, Y407, K409, T411 and K439; and the other polypeptide chain of the antibody heavy chain constant region comprises one or more mutations, relative to SEQ ID NO:26, at one or more positions selected from Q347, Y349, L351, S354, E356, E357, S364, T366, L368, K370, N390, K392, T394, D399, D401, F405, Y407, K409, T411, and K439, numbered according to the EU numbering system. In some embodiments, one polypeptide chain of the antibody heavy chain constant region comprises K360E and K409W substitutions relative to SEQ ID NO:26; and the other polypeptide chain of the antibody heavy chain constant region comprises Q347R, D399V and F405T substitutions relative to SEQ ID NO:26, numbered according to the EU numbering system.

In some embodiments, one polypeptide chain of the antibody heavy chain constant region comprises a Y349C substitution relative to SEQ ID NO:26; and the other polypeptide chain of the antibody heavy chain constant region comprises an S354C substitution relative to SEQ ID NO:26, numbered according to the EU numbering system.

Another aspect of the present application provides an antibody-drug conjugate comprising a protein as disclosed herein and a drug moiety. In some embodiments, the drug moiety is selected from the group consisting of auristatin, N-acetyl-γ calicheamicin, maytansinoid, pyrrolobenzodiazepine, and SN-38.

Another aspect of the present application provides an immunocytokine comprising an antigen-binding site as disclosed herein and a cytokine. In some embodiments, the cytokine is selected from the group consisting of IL-2, IL-4, IL-10, IL-12, IL-15, TNF, and IFNα.

Another aspect of the present application provides a bispecific T-cell engager comprising an antigen-binding site as disclosed herein and an antigen-binding site that binds CD3.

Another aspect of the present application provides a chimeric antigen receptor (CAR) comprising (a) an antigen-binding site as disclosed herein, (b) a transmembrane domain, and (c) an intracellular signaling domain. In some embodiments, the transmembrane domain is selected from the transmembrane regions of the alpha, beta or zeta chain of the T-cell receptor, CD28, CD3 epsilon, CD45, CD4, CD5, CD8, CD9, CD16, CD22, EGFR, CD37, CD64, CD80, CD86, CD134, CD137, CD152, and CD154. In some embodiments, the intracellular signaling domain comprises a primary signaling domain comprising a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In some embodiments, the intracellular signaling domain further comprises a costimulatory signaling domain comprising a functional signaling domain of a costimulatory receptor. In some embodiments, the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

Another aspect of the present application provides an isolated nucleic acid encoding a CAR as disclosed herein. Another aspect of the present application provides an expression vector comprising an isolated nucleic acid as disclosed herein. Another aspect of the present application provides an immune effector cell comprising the nucleic acid or the expression vector.

Another aspect of the present application provides an immune effector cell expressing a CAR as disclosed herein. In some embodiments, the immune effector cell is a T cell. In some embodiments, the T cell is a CD8⁺ T cell, a CD4⁺ T cell, a γδ T cell, or an NKT cell. In some embodiments, the immune effector cell is an NK cell.

Another aspect of the present application provides a pharmaceutical composition comprising a protein as disclosed herein, an antibody-drug conjugate as disclosed herein, an immunocytokine as disclosed herein, a bispecific T-cell engager as disclosed herein, or an immune effector cell as disclosed herein; and a pharmaceutically acceptable carrier.

Another aspect of the present application provides a method of treating cancer, comprising administering to a subject in need thereof an effective amount of a protein as disclosed herein, an antibody-drug conjugate as disclosed herein, an immunocytokine as disclosed herein, a bispecific T-cell engager as disclosed herein, or an immune effector cell as disclosed herein or a pharmaceutically composition as disclosed herein.

In some embodiments, the cancer is a solid tumor. In some embodiments, the cancer is lung cancer, breast cancer, kidney cancer, colorectal cancer, gastric cancer, brain cancer, glioma, bladder cancer, head and neck cancer, bladder cancer, pancreatic cancer, and liver cancer, cervical cancer, ovarian cancer or prostate cancer. In some embodiments, the cancer expresses EGFR.

In another aspect, the present application provides a purified antigen-binding site, protein, antibody-drug conjugate, immunocytokine, or bispecific T cell engager as disclosed herein. In some embodiments, the purified antigen-binding site, protein, antibody-drug conjugate, immunocytokine, or bispecific T cell engager is purified by a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.

These and other aspects and advantages of the antigen-binding sites described in the present application are illustrated by the following figures, detailed description and claims.

### DESCRIPTION OF THE DRAWINGS

The application can be more completely understood with reference to the following drawings.

FIG. 1 is a diagram showing the structural modeling of panitumumab having a S62R substitution in the VH (under the Chothia numbering scheme, in which the hydrogen bond between this Arg and the Asp at position 1 of the VL may contribute to stabilization of the VH-VL interface.

FIG. 2 is a diagram showing the structural modeling of panitumumab having a F87Y substitution in the VL (under the Chothia numbering scheme), in which the hydrogen bond between this Tyr and the Gln at position 39 of the VH may contribute to stabilization of the VH-VL interface.

FIG. 3 is a diagram showing the structural modeling of panitumumab having a D92R substitution in the VL, in which the van der Waals' contact between this Arg (in CDRL3) and the Tyr at position 32 of the VL (in CDRL1) may contribute to stabilization of the paratope.

FIG. 4 is an SPR sensorgram for a titration of EGFR-Protein-1 binding to human EGFR.

FIG. 5 is an SPR sensorgram for a titration of EGFR-Protein-2 binding to human EGFR.

FIG. 6 is an SPR sensorgram for a titration of EGFR-Protein-3 binding to human EGFR.

FIG. 7 is an SPR sensorgram for a titration of EGFR-Protein-4 binding to human EGFR.

FIGS. 8A, 8B, 8C, and 8D are plots showing the thermograms for EGFR-Protein-1 (FIG. 8A), EGFR-Protein-2 (FIG. 8B), EGFR-Protein-3 (FIG. 8C), and EGFR-Protein-4 (FIG. 8D) in PBS, pH 7.4, respectively, as determined by differential scanning calorimetry (DSC) analysis.

FIGS. 9A and 9B are plots showing the thermograms for EGFR-Protein-3 (FIG. 9A) and EGFR-Protein-4 (FIG. 9B), in 20 mM histidine, 250 mM trehalose, 0.01% PS80, pH 6.0, respectively, as determined by DSC analysis.

FIG. 10 is a plot showing the binding affinity of a series of concentrations of EGFR-Protein-1 (“EGFR1”), EGFR-Protein-2 (“EGFR2”), EGFR-Protein-3 (“EGFR3”), and panitumumab to EGFR-positive H2172 cancer cells.

FIG. 11 is a plot showing proliferation of an EGFR-positive cell line for 72 hours in the presence of a series of concentrations of EGFR-Protein-1 (“EGFR1”), EGFR-Protein-2 (“EGFR2”), EGFR-Protein-3 (“EGFR3”), panitumumab, and cetuximab.

### DETAILED DESCRIPTION

The present application provides antigen-binding sites that bind human EGFR. These antigen-binding sites bind various epitopes in an extracellular domain of EGFR. Proteins and protein conjugates containing such antigen-binding sites, for example, antibodies, antibody-drug conjugates, bispecific T-cell engagers (BiTEs), and immunocytokines, as well as immune effector cells (e.g., T cells) expressing a protein containing such an antigen-binding site (e.g., a chimeric antigen receptor (CAR)), are useful for treating EGFR-associated diseases such as cancer. The present application also provides pharmaceutical compositions comprising such proteins, protein conjugates, and immune effector cells, and therapeutic methods using such proteins, protein conjugates, immune effector cells, and pharmaceutical compositions, including for the treatment of cancer. Various aspects of the antigen-binding sites described in the present application are set forth in the sections below; however, aspects of the antigen-binding sites described in the present application described in one particular section are not to be limited to any particular section.

To facilitate an understanding of the present application, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of described in the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound described in the present application which, upon administration to a subject, is capable of providing a compound described in this application or an active metabolite or residue thereof. As is known to those of skill in the art, “salts” of the compounds described in the present application may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds described in the present application and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds described in the present application compounded with a suitable cation such as Na⁺, NH₄⁺, and NW₄⁺ (wherein W is a C₁₋₄ alkyl group), and the like.

For therapeutic use, salts of the compounds described in the present application are contemplated as being pharmaceutically acceptable. However, salts of acids and bases that are non-pharmaceutically acceptable may also find use, for example, in the preparation or purification of a pharmaceutically acceptable compound.

As used herein, EGFR (also known as epidermal growth factor receptor, ErbB-1, or HER1 in humans) refers to the protein of Uniprot Accession No. P00533 (humans) and related isoforms and orthologs.

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

Various features and aspects of the antigen-binding sites described in the application are discussed in more detail below.

### I. Antigen-Binding Site

In one aspect, the present application provides an antigen-binding site that binds human EGFR, wherein the antigen-binding site comprises a heavy chain variable domain (VH) and a light chain variable domain (VL) derived from panitumumab and having mutations in the VH and/or VL. Mutations that are contemplated, individually or in combination, in the VH and VL sequences described in the present application include S62R in the VH, D92R in the VL, and/or F87Y in the VL, under the Chothia numbering scheme. It has been discovered that these mutations increase thermostability of the antigen-binding site and retain its affinity to EGFR. The VH, VL, CDR, and scFv sequences of exemplary antigen-binding sites are listed in Table 1. The CDR sequences are identified according to the Chothia numbering scheme. Residues in bold, indicate mutated residues and italicized sequence indicates a polypeptide linker.

TABLE 1
Sequences of Exemplary Antigen-Binding Sites that Bind EGFR

| | Heavy chain variable domain | Light chain variable domain |
|---|---|---|
| EGFR binder | amino acid sequence | amino acid sequence |
| EGFR-binder-1 | QVQLQESGPGLVKPSETLSLTC | DIQMTQSPSSLSASVGDRVTI |
| (panitumumab) | TVSGGSVSSGDYYWTWIRQSP | TCQASQDISNYLNWYQQKPG |
| | GKGLEWIGHIYYSGNTNYNPS | KAPKLLIYDASNLETGVPSRF |
| | LKSRLTISIDTSKTQFSLKLSSV | SGSGSGTDFTFTISSLQPEDIA |
| | TAADTAIYYCVRDRVTGAFDI | TYFCQHFDHLPLAFGGGTKV |
| | WGQGTMVTVSS | EIK |
| | (SEQ ID NO: 1) | (SEQ ID NO: 5) |
| | CDR1: GGSVSSGDYYWT (SEQ | CDR1: QASQDISNYLN (SEQ |
| | ID NO: 2) | ID NO: 6) |
| | CDR2: HIYYSGNTNYNPSLKS | CDR2: LLIYDASNLET (SEQ |
| | (SEQ ID NO: 3) | ID NO: 7) |
| | CDR3: DRVTGAFDI (SEQ ID | CDR3: QHFDHLPLA (SEQ ID |
| | NO: 4) | NO: 8) |
| EGFR-scFv-1 | EGFR-scFv-1 (VL-VH): | |
| | DIQMTQSPSSLSASVGDRVTITCQASQDISNYLNWYQQKPGKAP | |
| | KLLIYDASNLETGVPSRFSGSGSGTDFTFTISSLQPEDIATYFCQH | |
| | FDHLPLAFGCGTKVEIKGGGGSGGGGSGGGGSGGGGSQVQLQ | |
| | ESGPGLVKPSETLSLTCTVSGGSVSSGDYYWTWIRQSPGKCLE | |
| | WIGHIYYSGNTNYNPSLKSRLTISIDTSKTQFSLKLSSVTAADTAI | |
| | YYCVRDRVTGAFDIWGQGTMVTVSS (SEQ ID NO: 9) | |
| | EGFR-scFv-1 (VH-VL): | |
| | QVQLQESGPGLVKPSETLSLTCTVSGGSVSSGDYYWTWIRQSP | |
| | GKCLEWIGHIYYSGNTNYNPSLKSRLTISIDTSKTQFSLKLSSVT | |
| | AADTAIYYCVRDRVTGAFDIWGQGTMVTVSSGGGGSGGGGSG | |
| | GGGSGGGGSDIQMTQSPSSLSASVGDRVTITCQASQDISNYLNW | |
| | YQQKPGKAPKLLIYDASNLETGVPSRFSGSGSGTDFTFTISSLQP | |
| | EDIATYFCQHFDHLPLAFGCGTKVEIK (SEQ ID NO: 10) | |
| EGFR-binder-2 | QVQLQESGPGLVKPSETLSLTC | DIQMTQSPSSLSASVGDRVTI |
| | TVSGGSVSSGDYYWTWIRQSP | TCQASQDISNYLNWYQQKPG |
| | GKGLEWIGHIYYSGNTNYNPR | KAPKLLIYDASNLETGVPSRF |
| | LKSRLTISIDTSKTQFSLKLSSV | SGSGSGTDFTFTISSLQPEDIA |
| | TAADTAIYYCVRDRVTGAFDI | TYYCQHFDHLPLAFGGGTK |
| | WGQGTMVTVSS (SEQ ID | VEIK (SEQ ID NO: 13) |
| | NO: 11) | CDR1: QASQDISNYLN (SEQ |
| | CDR1: GGSVSSGDYYWT (SEQ | ID NO: 6) |
| | ID NO: 2) | CDR2: LLIYDASNLET (SEQ |
| | CDR2: HIYYSGNTNYNPRLKS | ID NO: 7) |
| | (SEQ ID NO: 12) | CDR3: QHFDHLPLA (SEQ ID |
| | CDR3: DRVTGAFDI (SEQ ID | NO: 8) |
| | NO: 4) | |
| EGFR-scFv-2 | EGFR-scFv-2 (VL-VH): | |
| | DIQMTQSPSSLSASVGDRVTITCQASQDISNYLNWYQQKPGKAP | |
| | KLLIYDASNLETGVPSRFSGSGSGTDFTFTISSLQPEDIATYYCQ | |
| | HFDHLPLAFGCGTKVEIKGGGGSGGGGSGGGGSGGGGSQVQL | |
| | QESGPGLVKPSETLSLTCTVSGGSVSSGDYYWTWIRQSPGKCL | |
| | EWIGHIYYSGNTNYNPRLKSRLTISIDTSKTQFSLKLSSVTAADT | |
| | AIYYCVRDRVTGAFDIWGQGTMVTVSS (SEQ ID NO: 14) | |
| | EGFR-scFv-2 (VH-VL): | |
| | QVQLQESGPGLVKPSETLSLTCTVSGGSVSSGDYYWTWIRQSP | |
| | GKCLEWIGHIYYSGNTNYNPRLKSRLTISIDTSKTQFSLKLSSVT | |
| | AADTAIYYCVRDRVTGAFDIWGQGTMVTVSSGGGGSGGGGSG | |
| | GGGSGGGGSDIQMTQSPSSLSASVGDRVTITCQASQDISNYLNW | |
| | YQQKPGKAPKLLIYDASNLETGVPSRFSGSGSGTDFTFTISSLQP | |
| | EDIATYYCQHFDHLPLAFGCGTKVEIK (SEQ ID NO: 15) | |
| EGFR-binder-3 | QVQLQESGPGLVKPSETLSLTC | DIQMTQSPSSLSASVGDRVTI |
| | TVSGGSVSSGDYYWTWIRQSP | TCQASQDISNYLNWYQQKPG |
| | GKGLEWIGHIYYSGNTNYNPR | KAPKLLIYDASNLETGVPSRF |
| | LKSRLTISIDTSKTQFSLKLSSV | SGSGSGTDFTFTISSLQPEDIA |
| | TAADTAIYYCVRDRVTGAFDI | TYYCQHFRHLPLAFGGGTK |
| | WGQGTMVTVSS (SEQ ID | VEIK (SEQ ID NO: 16) |
| | NO: 11) | CDR1: QASQDISNYLN (SEQ |
| | CDR1: GGSVSSGDYYWT (SEQ | ID NO: 6) |
| | ID NO: 2) | CDR2: LLIYDASNLET (SEQ |
| | CDR2: HIYYSGNTNYNPRLKS | ID NO: 7) |
| | (SEQ ID NO: 12) | CDR3: QHFRHLPLA (SEQ ID |
| | CDR3: DRVTGAFDI (SEQ ID | NO: 17) |
| | NO: 4) | |
| EGFR-binder- | QVQLQESGPGLVKPSETLSLTC | DIQMTQSPSSLSASVGDRVTI |
| 3(2) | TVSGGSVSSGDYYWTWIRQSP | TCQASQDISNYLNWYQQKPG |
| | GKCLEWIGHIYYSGNTNYNPR | KAPKLLIYDASNLETGVPSRF |
| | LKSRLTISIDTSKTQFSLKLSSV | SGSGSGTDFTFTISSLQPEDIA |
| | TAADTAIYYCVRDRVTGAFDI | TYYCQHFRHLPLAFGCGTK |
| | WGQGTMVTVSS (SEQ ID | VEIK (SEQ ID NO: 33) |
| | NO: 32) | CDR1: QASQDISNYLN (SEQ |
| | CDR1: GGSVSSGDYYWT (SEQ | ID NO: 6) |
| | ID NO: 2) | CDR2: LLIYDASNLET (SEQ |
| | CDR2: HIYYSGNTNYNPRLKS | ID NO: 7) |
| | (SEQ ID NO: 12) | CDR3: QHFRHLPLA (SEQ ID |
| | CDR3: DRVTGAFDI (SEQ ID | NO: 17) |
| | NO: 4) | |
| EGFR-scFv-3 | EGFR-scFv-3 (VL-VH): | |
| | DIQMTQSPSSLSASVGDRVTITCQASQDISNYLNWYQQKPGKAP | |
| | KLLIYDASNLETGVPSRFSGSGSGTDFTFTISSLQPEDIATYYCQ | |
| | HFRHLPLAFGCGTKVEIKGGGGSGGGGSGGGGSGGGGSQVQL | |
| | QESGPGLVKPSETLSLTCTVSGGSVSSGDYYWTWIRQSPGKCL | |
| | EWIGHIYYSGNTNYNPRLKSRLTISIDTSKTQFSLKLSSVTAADT | |
| | AIYYCVRDRVTGAFDIWGQGTMVTVSS (SEQ ID NO: 18) | |
| | EGFR-scFv-3 (VH-VL): | |
| | QVQLQESGPGLVKPSETLSLTCTVSGGSVSSGDYYWTWIRQSP | |
| | GKCLEWIGHIYYSGNTNYNPRLKSRLTISIDTSKTQFSLKLSSVT | |
| | AADTAIYYCVRDRVTGAFDIWGQGTMVTVSSGGGGSGGGGSG | |
| | GGGSGGGGSDIQMTQSPSSLSASVGDRVTITCQASQDISNYLNW | |
| | YQQKPGKAPKLLIYDASNLETGVPSRFSGSGSGTDFTFTISSLQP | |
| | EDIATYYCQHFRHLPLAFGCGTKVEIK (SEQ ID NO: 19) | |
| EGFR-binder-4 | QVQLQESGPGLVKPSETLSLTC | DIQMTQSPSSLSASVGDRVTI |
| | TVSGGSVSSGDYYWTWIRQSP | TCQASQDISNYLNWYQQKPG |
| | GKGLEWIGHIYYSGNTNYNPS | KAPKLLIYDASNLETGVPSRF |
| | LKSRLTISIDTSKTQFSLKLSSV | SGSGSGTDFTFTISSLQPEDIA |
| | TAADTAIYYCVRDRVTGAFDI | TYYCQHFRHLPLAFGGGTK |
| | WGQGTMVTVSS (SEQ ID | VEIK (SEQ ID NO: 16) |
| | NO: 1) | CDR1: QASQDISNYLN (SEQ |
| | CDR1: GGSVSSGDYYWT (SEQ | ID NO: 6) |
| | ID NO: 2) | CDR2: LLIYDASNLET (SEQ |
| | CDR2: HIYYSGNTNYNPSLKS | ID NO: 7) |
| | (SEQ ID NO: 3) | CDR3: QHFRHLPLA (SEQ ID |
| | CDR3: DRVTGAFDI (SEQ ID | NO: 17) |
| | NO: 4) | |
| EGFR-scFv-4 | EGFR-scFv-4 (VL-VH): | |
| | DIQMTQSPSSLSASVGDRVTITCQASQDISNYLNWYQQKPGKAP | |
| | KLLIYDASNLETGVPSRFSGSGSGTDFTFTISSLQPEDIATYYCQ | |
| | HFRHLPLAFGCGTKVEIKGGGGSGGGGSGGGGSGGGGSQVQL | |
| | QESGPGLVKPSETLSLTCTVSGGSVSSGDYYWTWIRQSPGKCL | |
| | EWIGHIYYSGNTNYNPSLKSRLTISIDTSKTQFSLKLSSVTAADT | |
| | AIYYCVRDRVTGAFDIWGQGTMVTVSS (SEQ ID NO: 20) | |
| | EGFR-scFv-4 (VH-VL): | |
| | QVQLQESGPGLVKPSETLSLTCTVSGGSVSSGDYYWTWIRQSP | |
| | GKCLEWIGHIYYSGNTNYNPSLKSRLTISIDTSKTQFSLKLSSVT | |
| | AADTAIYYCVRDRVTGAFDIWGQGTMVTVSSGGGGSGGGGSG | |
| | GGGSGGGGSDIQMTQSPSSLSASVGDRVTITCQASQDISNYLNW | |
| | YQQKPGKAPKLLIYDASNLETGVPSRFSGSGSGTDFTFTISSLQP | |
| | EDIATYYCQHFRHLPLAFGCGTKVEIK (SEQ ID NO: 21) | |
| Consensus | QVQLQESGPGLVKPSETLSLTC | DIQMTQSPSSLSASVGDRVTI |
| Sequence | TVSGGSVSSGDYYWTWIRQSP | TCQASQDISNYLNWYQQKPG |
| (EGFR-binder-3 | GKGLEWIGHIYYSGNTNYNPX | KAPKLLIYDASNLETGVPSRF |
| and EGFR- | LKSRLTISIDTSKTQFSLKLSSV | SGSGSGTDFTFTISSLQPEDIA |
| binder-4) | TAADTAIYYCVRDRVTGAFDI | TYYCQHFRHLPLAFGGGTK |
| | WGQGTMVTVSS | VEIK (SEQ ID NO: 16) |
| | where X is R or S | CDR1: QASQDISNYLN (SEQ |
| | (SEQ ID NO: 22) | ID NO: 6) |
| | CDR1: GGSVSSGDYYWT (SEQ | CDR2: LLIYDASNLET (SEQ |
| | ID NO: 2) | ID NO: 7) |
| | CDR2: HIYYSGNTNYNPXLKS | CDR3: QHFRHLPLA (SEQ ID |
| | where X is R or S (SEQ ID NO: 23) | NO: 17) |
| | CDR3: DRVTGAFDI (SEQ ID | |
| | NO: 4) | |

| | |
|---|---|
| Consensus | scFv-309 (VL-VH): |
| Sequence | DIQMTQSPSSLSASVGDRVTITCQASQDISNYLNWYQQKPGKAP |
| (EGFR-scFv-3 | KLLIYDASNLETGVPSRFSGSGSGTDFTFTISSLQPEDIATYYCQ |
| and EGFR-scFv- | HFRHLPLAFGCGTKVEIKGGGGSGGGGSGGGGSGGGGSQVQL |
| 4) | QESGPGLVKPSETLSLTCTVSGGSVSSGDYYWTWIRQSPGKCLE |
| | WIGHIYYSGNTNYNPXLKSRLTISIDTSKTQFSLKLSSVTAADTA |
| | IYYCVRDRVTGAFDIWGQGTMVTVSS |
| | where X is R or S |
| | (SEQ ID NO: 24) |
| | scFv-310 (VH-VL): |
| | QVQLQESGPGLVKPSETLSLTCTVSGGSVSSGDYYWTWIRQSP |
| | GKCLEWIGHIYYSGNTNYNPXLKSRLTISIDTSKTQFSLKLSSVT |
| | AADTAIYYCVRDRVTGAFDIWGQGTMVTVSSGGGGSGGGGSG |
| | GGGSGGGGSDIQMTQSPSSLSASVGDRVTITCQASQDISNYLNW |
| | YQQKPGKAPKLLIYDASNLETGVPSRFSGSGSGTDFTFTISSLQP |
| | EDIATYYCQHFRHLPLAFGCGTKVEIK |
| | where X is R or S |
| | (SEQ ID NO: 25) |

In certain embodiments, the amino acid sequence of the antigen-binding site comprises an additional arginine (R) residue at the C-terminus of the VL (e.g., SEQ TD NO: 5, 13, or 16). In certain embodiments, the antigen-binding site is present as an scFv, wherein the scFv comprises a VL amino acid sequence that contains an additional arginine (R) residue at the C-terminus of the VL amino acid sequence.

In certain embodiments, the amino acid sequence of the antigen-binding site comprises one or more mutations relative to the sequence of panitumumab selected from S62R in the VH, D92R in the VL, and F87Y in the VL, under the Chothia numbering scheme. In certain embodiments, the antigen-binding site that binds EGFR (e.g., human EGFR) comprises an antibody heavy chain variable domain (VH) that comprises an amino acid sequence at least 90% (e.g., at least 91%, at least 92%, at least 93%, at least 94%, at least 9500, at least 9600, at least 970%, at least 98%, or at least 990%) identical to the VH of EGFR-binder-1 as disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, or at least 99%) identical to the VL of EGFR-binder-1 as disclosed in Table 1. In certain embodiments, the antigen-binding site that binds EGFR (e.g., human EGFR) comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of EGFR-binder-2, EGFR-binder-3, or EGFR-binder-4 as disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of EGFR-binder-2, EGFR-binder-3, or EGFR-binder-4 as disclosed in Table 1. In certain embodiments, the antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences of EGFR-binder-2, EGFR-binder-3, or EGFR-binder-4 as disclosed in Table 1. In certain embodiments, the antigen-binding site comprises the heavy chain CDR1, CDR2, and CDR3 and the light chain CDR1, CDR2, and CDR3 of EGFR-binder-2, EGFR-binder-3, or EGFR-binder-4 as disclosed in Table 1.

In certain embodiments, the amino acid sequence of the antigen-binding site comprises S62R in the VH and F87Y in the VL mutations relative to the sequence of panitumumab, under the Chothia numbering scheme. In certain embodiments, the antigen-binding site that binds EGFR comprises a VH that comprises an amino acid sequence at least 90% (e.g., at least 91%, at least 92%, at least 93%, at least 94%, at least 95%, at least 96%, at least 97%, at least 98%, or at least 99%) identical to SEQ ID NO:1, and a VL that comprises an amino acid sequence at least 90% (e.g., at least 91%, at least 92%, at least 93%, at least 94%, at least 95%, at least 96%, at least 97%, at least 98%, or at least 99%) identical to SEQ ID NO:5. In certain embodiments, the antigen-binding site that binds EGFR comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:11, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:13. In certain embodiments, the antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences of EGFR-binder-2 disclosed in Table 1. In certain embodiments, the VH comprises CDR1, CDR2, and CDR3 comprising the amino acid sequences of SEQ ID NOs: 2, 12, and 4, respectively. In certain embodiments, the VL comprises CDR1, CDR2, and CDR3 comprising the amino acid sequences of SEQ ID NOs: 6, 7, and 8, respectively. In certain embodiments, the antigen-binding site comprises (a) a VH that comprises CDR1, CDR2, and CDR3 comprising the amino acid sequences of SEQ ID NOs: 2, 12, and 4, respectively; and (b) a VL that comprises CDR1, CDR2, and CDR3 comprising the amino acid sequences of SEQ ID NOs: 6, 7, and 8, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 14 or 15. In certain embodiments, the antigen-binding site is present as an scFv, wherein the scFv comprises an amino acid sequence identical to SEQ ID NO: 14 or 15. In certain embodiments, the antigen-binding site is present as an scFv, wherein the scFv comprises an amino acid sequence identical to SEQ ID NO:14. In certain embodiments, the antigen-binding site is present as an scFv, wherein the scFv comprises an amino acid sequence identical to SEQ ID NO:15.

In certain embodiments, the amino acid sequence of the antigen-binding site comprises S62R in the VH, F87Y in the VL, and D92R in the VL mutations relative to the sequence of panitumumab, under the Chothia numbering scheme. In certain embodiments, the antigen-binding site that binds EGFR comprises a VH that comprises an amino acid sequence at least 90% (e.g., at least 91%, at least 92%, at least 93%, at least 94%, at least 95%, at least 96%, at least 97%, at least 98%, or at least 99%) identical to SEQ ID NO:1, and a VL that comprises an amino acid sequence at least 90% (e.g., at least 91%, at least 92%, at least 93%, at least 94%, at least 95%, at least 96%, at least 97%, at least 98%, or at least 99%) identical to SEQ ID NO:5. In certain embodiments, the antigen-binding site that binds EGFR comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:11, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:16. In certain embodiments, the antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences of EGFR-binder-3 disclosed in Table 1. In certain embodiments, the VH comprises CDR1, CDR2, and CDR3 comprising the amino acid sequences of SEQ ID NOs: 2, 12, and 4, respectively. In certain embodiments, the VL comprises CDR1, CDR2, and CDR3 comprising the amino acid sequences of SEQ ID NOs: 6, 7, and 17, respectively. In certain embodiments, the antigen-binding site comprises (a) a VH that comprises CDR1, CDR2, and CDR3 comprising the amino acid sequences of SEQ ID NOs: 2, 12, and 4, respectively; and (b) a VL that comprises CDR1, CDR2, and CDR3 comprising the amino acid sequences of SEQ ID NOs: 6, 7, and 17, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 18 or 19. In certain embodiments, the antigen-binding site is present as an scFv, wherein the scFv comprises an amino acid sequence identical to SEQ ID NO: 18 or 19. In certain embodiments, the antigen-binding site is present as an scFv, wherein the scFv comprises an amino acid sequence identical to SEQ ID NO:18. In certain embodiments, the antigen-binding site is present as an scFv, wherein the scFv comprises an amino acid sequence identical to SEQ ID NO:19.

In certain embodiments, the amino acid sequence of the antigen-binding site comprises F87Y in the VL and D92R in the VL mutations relative to the sequence of panitumumab, under the Chothia numbering scheme. In certain embodiments, the antigen-binding site that binds EGFR comprises a VH that comprises an amino acid sequence at least 90% (e.g., at least 91%, at least 92%, at least 93%, at least 94%, at least 95%, at least 96%, at least 97%, at least 98%, or at least 99%) identical to SEQ ID NO:1, and a VL that comprises an amino acid sequence at least 90% (e.g., at least 91%, at least 92%, at least 93%, at least 94%, at least 95%, at least 96%, at least 97%, at least 98%, or at least 99%) identical to SEQ ID NO:5. In certain embodiments, the antigen-binding site that binds EGFR comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:1, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:16. In certain embodiments, the antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences of EGFR-binder-4 disclosed in Table 1. In certain embodiments, the VH comprises CDR1, CDR2, and CDR3 comprising the amino acid sequences of SEQ ID NOs: 2, 3, and 4, respectively. In certain embodiments, the VL comprises CDR1, CDR2, and CDR3 comprising the amino acid sequences of SEQ ID NOs: 6, 7, and 17, respectively. In certain embodiments, the antigen-binding site comprises (a) a VH that comprises CDR1, CDR2, and CDR3 comprising the amino acid sequences of SEQ ID NOs: 2, 3, and 4, respectively; and (b) a VL that comprises CDR1, CDR2, and CDR3 comprising the amino acid sequences of SEQ ID NOs: 6, 7, and 17, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 20 or 21. In certain embodiments, the antigen-binding site is present as an scFv, wherein the scFv comprises an amino acid sequence identical to SEQ ID NO: 20 or 21. In certain embodiments, the antigen-binding site is present as an scFv, wherein the scFv comprises an amino acid sequence identical to SEQ ID NO:20. In certain embodiments, the antigen-binding site is present as an scFv, wherein the scFv comprises an amino acid sequence identical to SEQ ID NO:21.

In certain embodiments, the amino acid sequence of the antigen-binding site comprises F87Y in the VL and D92R in the VL, and optionally S62R in the VH mutations relative to the sequence of panitumumab, under the Chothia numbering scheme. In certain embodiments, the antigen-binding site that binds EGFR comprises a VH that comprises an amino acid sequence at least 90% (e.g., at least 91%, at least 92%, at least 93%, at least 94%, at least 95%, at least 96%, at least 97%, at least 98%, or at least 99%) identical to SEQ ID NO:1, and a VL that comprises an amino acid sequence at least 90% (e.g., at least 91%, at least 92%, at least 93%, at least 94%, at least 95%, at least 96%, at least 97%, at least 98%, or at least 99%) identical to SEQ ID NO:5. In certain embodiments, the antigen-binding site that binds EGFR comprises a heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:22, and a light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:16. In certain embodiments, the antigen-binding site comprises the heavy chain CDR1, CDR2, and CDR3 and the light chain CDR1, CDR2, and CDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the consensus VH and VL sequences disclosed in Table 1. In certain embodiments, the VH comprises CDR1, CDR2, and CDR3 comprising the amino acid sequences of SEQ ID NOs: 2, 23, and 4, respectively. In certain embodiments, the VL comprises CDR1, CDR2, and CDR3 comprising the amino acid sequences of SEQ ID NOs: 6, 7, and 17, respectively. In certain embodiments, the antigen-binding site comprises (a) a VH that comprises CDR1, CDR2, and CDR3 comprising the amino acid sequences of SEQ ID NOs: 2, 23, and 4, respectively; and (b) a VL that comprises CDR1, CDR2, and CDR3 comprising the amino acid sequences of SEQ ID NOs: 6, 7, and 17, respectively. In certain embodiments, the antigen-binding site is present as an scFv, wherein the scFv comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 24 or 25. In certain embodiments, the antigen-binding site is present as an scFv, wherein the scFv comprises an amino acid sequence identical to SEQ ID NO: 24 or 25. In certain embodiments, the antigen-binding site is present as an scFv, wherein the scFv comprises an amino acid sequence identical to SEQ ID NO:24. In certain embodiments, the antigen-binding site is present as an scFv, wherein the scFv comprises an amino acid sequence identical to SEQ ID NO:25.

Alternatively, novel antigen-binding sites that can bind to EGFR can be identified by screening for binding to the amino acid sequence of EGFR, an isoform thereof, a variant thereof, a mature extracellular fragment thereof or a fragment containing a domain of EGFR.

TABLE 2
Sequences for exemplary EGFR Isoforms

| EGFR Isoform | Amino Acid Sequence |
|---|---|
| 1 | MRPSGTAGAALLALLAALCPASRALEEKKVCQGTSNKLTQLGT |
| (Uniprot ID No.: | FEDHFLSLQRMFNNCEVVLGNLEITYVQRNYDLSFLKTIQEVAG |
| P00533-1) | YVLIALNTVERIPLENLQIIRGNMYYENSYALAVLSNYDANKTG |
| | LKELPMRNLQEILHGAVRFSNNPALCNVESIQWRDIVSSDFLSN |
| | MSMDFQNHLGSCQKCDPSCPNGSCWGAGEENCQKLTKIICAQ |
| | QCSGRCRGKSPSDCCHNQCAAGCTGPRESDCLVCRKFRDEATC |
| | KDTCPPLMLYNPTTYQMDVNPEGKYSFGATCVKKCPRNYVVT |
| | DHGSCVRACGADSYEMEEDGVRKCKKCEGPCRKVCNGIGIGE |
| | FKDSLSINATNIKHFKNCTSISGDLHILPVAFRGDSFTHTPPLDPQ |
| | ELDILKTVKEITGFLLIQAWPENRTDLHAFENLEIIRGRTKQHGQ |
| | FSLAVVSLNITSLGLRSLKEISDGDVIISGNKNLCYANTINWKKL |
| | FGTSGQKTKIISNRGENSCKATGQVCHALCSPEGCWGPEPRDC |
| | VSCRNVSRGRECVDKCNLLEGEPREFVENSECIQCHPECLPQA |
| | MNITCTGRGPDNCIQCAHYIDGPHCVKTCPAGVMGENNTLVW |
| | KYADAGHVCHLCHPNCTYGCTGPGLEGCPTNGPKIPSIATGMV |
| | GALLLLLVVALGIGLFMRRRHIVRKRTLRRLLQERELVEPLTPS |
| | GEAPNQALLRILKETEFKKIKVLGSGAFGTVYKGLWIPEGEKVK |
| | IPVAIKELREATSPKANKEILDEAYVMASVDNPHVCRLLGICLTS |
| | TVQLITQLMPFGCLLDYVREHKDNIGSQYLLNWCVQIAKGMN |
| | YLEDRRLVHRDLAARNVLVKTPQHVKITDFGLAKLLGAEEKEY |
| | HAEGGKVPIKWMALESILHRIYTHQSDVWSYGVTVWELMTFG |
| | SKPYDGIPASEISSILEKGERLPQPPICTIDVYMIMVKCWMIDAD |
| | SRPKFRELIIEFSKMARDPQRYLVIQGDERMHLPSPTDSNFYRAL |
| | MDEEDMDDVVDADEYLIPQQGFFSSPSTSRTPLLSSLSATSNNS |
| | TVACIDRNGLQSCPIKEDSFLQRYSSDPTGALTEDSIDDTFLPVP |
| | EYINQSVPKRPAGSVQNPVYHNQPLNPAPSRDPHYQDPHSTAV |
| | GNPEYLNTVQPTCVNSTFDSPAHWAQKGSHQISLDNPDYQQDF |
| | FPKEAKPNGIFKGSTAENAEYLRVAPQSSEFIGA |
| | (SEQ ID NO: 27) |
| 2 | MRPSGTAGAALLALLAALCPASRALEEKKVCQGTSNKLTQLGT |
| (Uniprot ID No.: | FEDHFLSLQRMFNNCEVVLGNLEITYVQRNYDLSFLKTIQEVAG |
| P00533-2) | YVLIALNTVERIPLENLQIIRGNMYYENSYALAVLSNYDANKTG |
| | LKELPMRNLQEILHGAVRFSNNPALCNVESIQWRDIVSSDFLSN |
| | MSMDFQNHLGSCQKCDPSCPNGSCWGAGEENCQKLTKIICAQ |
| | QCSGRCRGKSPSDCCHNQCAAGCTGPRESDCLVCRKFRDEATC |
| | KDTCPPLMLYNPTTYQMDVNPEGKYSFGATCVKKCPRNYVVT |
| | DHGSCVRACGADSYEMEEDGVRKCKKCEGPCRKVCNGIGIGE |
| | FKDSLSINATNIKHFKNCTSISGDLHILPVAFRGDSFTHTPPLDPQ |
| | ELDILKTVKEITGLS |
| | (SEQ ID NO: 28) |
| 3 | MRPSGTAGAALLALLAALCPASRALEEKKVCQGTSNKLTQLGT |
| (Uniprot ID No.: | FEDHFLSLQRMFNNCEVVLGNLEITYVQRNYDLSFLKTIQEVAG |
| P00533-3) | YVLIALNTVERIPLENLQIIRGNMYYENSYALAVLSNYDANKTG |
| | LKELPMRNLQEILHGAVRFSNNPALCNVESIQWRDIVSSDFLSN |
| | MSMDFQNHLGSCQKCDPSCPNGSCWGAGEENCQKLTKIICAQ |
| | QCSGRCRGKSPSDCCHNQCAAGCTGPRESDCLVCRKFRDEATC |
| | KDTCPPLMLYNPTTYQMDVNPEGKYSFGATCVKKCPRNYVVT |
| | DHGSCVRACGADSYEMEEDGVRKCKKCEGPCRKVCNGIGIGE |
| | FKDSLSINATNIKHFKNCTSISGDLHILPVAFRGDSFTHTPPLDPQ |
| | ELDILKTVKEITGFLLIQAWPENRTDLHAFENLEIIRGRTKQHGQ |
| | FSLAVVSLNITSLGLRSLKEISDGDVIISGNKNLCYANTINWKKL |
| | FGTSGQKTKIISNRGENSCKATGQVCHALCSPEGCWGPEPRDC |
| | VSCRNVSRGRECVDKCNLLEGEPREFVENSECIQCHPECLPQA |
| | MNITCTGRGPDNCIQCAHYIDGPHCVKTCPAGVMGENNTLVW |
| | KYADAGHVCHLCHPNCTYGPGNESLKAMLFCLFKLSSCNQSN |
| | DGSVSHQSGSPAAQESCLGWIPSLLPSEFQLGWGGCSHLHAWP |
| | SASVIITASSCH |
| | (SEQ ID NO: 29) |
| 4 | MRPSGTAGAALLALLAALCPASRALEEKKVCQGTSNKLTQLGT |
| (Uniprot ID No.: | FEDHFLSLQRMFNNCEVVLGNLEITYVQRNYDLSFLKTIQEVAG |
| P00533-4) | YVLIALNTVERIPLENLQIIRGNMYYENSYALAVLSNYDANKTG |
| | LKELPMRNLQEILHGAVRFSNNPALCNVESIQWRDIVSSDFLSN |
| | MSMDFQNHLGSCQKCDPSCPNGSCWGAGEENCQKLTKIICAQ |
| | QCSGRCRGKSPSDCCHNQCAAGCTGPRESDCLVCRKFRDEATC |
| | KDTCPPLMLYNPTTYQMDVNPEGKYSFGATCVKKCPRNYVVT |
| | DHGSCVRACGADSYEMEEDGVRKCKKCEGPCRKVCNGIGIGE |
| | FKDSLSINATNIKHFKNCTSISGDLHILPVAFRGDSFTHTPPLDPQ |
| | ELDILKTVKEITGFLLIQAWPENRTDLHAFENLEIIRGRTKQHGQ |
| | FSLAVVSLNITSLGLRSLKEISDGDVIISGNKNLCYANTINWKKL |
| | FGTSGQKTKIISNRGENSCKATGQVCHALCSPEGCWGPEPRDC |
| | VSCRNVSRGRECVDKCNLLEGEPREFVENSECIQCHPECLPQA |
| | MNITCTGRGPDNCIQCAHYIDGPHCVKTCPAGVMGENNTLVW |
| | KYADAGHVCHLCHPNCTYGS |
| | (SEQ ID NO: 30) |

In some embodiments, novel antigen-binding sites that can bind to EGFR can be identified by screening for binding to the amino acid sequence defined by SEQ ID NOs: 27-30, a variant thereof, a mature extracellular fragment thereof or a fragment containing a domain of EGFR.

In certain embodiments, the antigen-binding site binds human EGFR or the extracellular region thereof at a K_{D} value less than or equal to (affinity greater than or equal to) 10 nM, 9 nM, 8 nM, 7 nM, 6 nM, 5 nM, or 4 nM. In certain embodiments, the antigen binding site of the present application binds human EGFR or the extracellular region thereof at a K_{D} value less than or equal to (affinity greater than or equal to) 4 nM. In certain embodiments, an antigen-binding site of the present application binds human EGFR or the extracellular region thereof at a K_{D} value less than or equal to (affinity greater than or equal to) about 2.0 nM, 2.1 nM, 2.2 nM, 2.3 nM, 2.4 nM, 2.5 nM, 2.6 nM, 2.7 nM, 2.8 nM, 2.9 nM, 3.0 nM, 3.1 nM, 3.2 nM, 3.3 nM, 3.4 nM, 3.5 nM, 3.6 nM, 3.7 nM, 3.8 nM, 3.9 nM, 4.0 nM, 4.1 nM, 4.2 nM, 4.3 nM, 4.4 nM, 4.5 nM, 4.6 nM, 4.7 nM, 4.8 nM, 4.9 nM or 5.0 nM. In certain embodiments, an antigen-binding site of the present application binds human EGFR or the extracellular region thereof at a K_{D} value in the range of about 1.0-3.5 nM, 1.0-4.0 nM, 1.0-4.5 nM, 1.0-5.0 nM, 1.5-3.5 nM, 1.5-4.0 nM, 1.5-4.5 nM, 1.5-5.0 nM, 2.0-3.5 nM, 2.0-4.0 nM, 2.0-4.5 nM, 2.0-5.0 nM, 2.5-3.5 nM, 2.5-4.0 nM, 2.5-4.5 nM, 2.5-5.0 nM, 3.0-3.5 nM, 3.0-4.0 nM, 3.0-4.5 nM, or 3.0-5.0 nM. These K_{D} values are as measured using standard binding assays, for example, surface plasmon resonance or bio-layer interferometry. In certain embodiments the antibody binds EGFR from a body fluid, tissue and/or cell of a subject.

In certain embodiments, the antigen-binding site binds rhesus macaque EGFR or the extracellular region thereof at a K_{D} value less than or equal to (affinity greater than or equal to) 10 nM, 9 nM, 8 nM, 7 nM, 6 nM, 5 nM, or 4 nM. In certain embodiments, an antigen-binding site of the present application binds rhesus macaque EGFR or the extracellular region thereof at a K_{D} value in the range of about 1-10 nM, 1-6 nM, 2-10 nM, 2-6 nM, 3-10 nM, 3-6 nM, 4-10 nM, 4-6 nM, 5-10 nM, or 5-6 nM. These K_{D} values are as measured using standard binding assays, for example, surface plasmon resonance or bio-layer interferometry. In certain embodiments the antibody binds EGFR from a body fluid, tissue and/or cell of a subject.

In certain embodiments, the antigen-binding site has greater thermostability than a corresponding antigen-binding site having the VH and VL sequences of SEQ ID NOs: 1 and 5, respectively, wherein the antigen-binding site does not comprise a G44C mutation in the VH and a G100C mutation in the VL. In certain embodiments, the antigen-binding disclosed herein has greater thermostability than a corresponding antigen-binding site having an amino acid sequence of SEQ ID NO:9 or 10, wherein the antigen-binding site takes an scFv format in the VL-VH or VH-VL orientation, respectively. Methods of measuring thermostability include but are not limited to differential scanning calorimetry (DSC), for example, as disclosed in Example 3 below. In certain embodiments, where the antigen-binding site takes an scFv format in the VL-VH orientation, a melting temperature of the antigen-binding site (e.g., Tₒₙₛₑₜ or Tₘ₁, as measured by DSC) is higher than the corresponding melting temperature of an scFv having the amino acid sequence of SEQ ID NO:9 by at least 1° C., 2° C., 3° C., 4° C., 5° C., or 6° C. In certain embodiments, where the antigen-binding site takes an scFv format in the VH-VL orientation, a melting temperature of the antigen-binding site (e.g., Tₒₙₛₑₜ or Tₘ₁, as measured by DSC) is higher than the corresponding melting temperature of an scFv having the amino acid sequence of SEQ ID NO:10 by at least 1° C., 2° C., 3° C., 4° C., 5° C., or 6° C.

Proteins with Antigen-Binding Sites

An antigen-binding site disclosed herein can be present in an antibody or antigen-binding fragment thereof. The antibody can be a monoclonal antibody, a chimeric antibody, a diabody, a Fab fragment, a Fab′ fragment, or F(ab′)₂ fragment, an Fv, a bispecific antibody, a bispecific Fab2, a bispecific (mab)2, a humanized antibody, an artificially-generated human antibody, bispecific T-cell engager, bispecific NK cell engager, a single chain antibody (e.g., single-chain Fv fragment or scFv), triomab, knobs-into-holes (kih) IgG with common light chain, crossmab, ortho-Fab IgG, DVD-Ig, 2 in 1-IgG, IgG-scFv, scFv2-Fc, bi-nanobody, tandAb, dual-affinity retargeting antibody (DART), DART-Fc, scFv-HSA-scFv (where HSA=human serum albumin), or dock-and-lock (DNL)-Fab3.

In certain embodiments, an antigen-binding site disclosed herein is linked to an amino acid sequence at least 90% (e.g., 90%, 91%, 92%, 93%, 94%, 95%, 96%, 97%, 98%, 99%, or 100%) identical to an antibody constant region, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, an antigen-binding site disclosed herein can be linked to a (e.g., human) light chain constant region chosen from, e.g., the light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the antigen-binding site is linked to an IgG constant region including hinge, CH2 and CH3 domains with or without a CH1 domain. In some embodiments, the amino acid sequence of the constant region is at least 90% (e.g., 90%, 91%, 92%, 93%, 94%, 95%, 96%, 97%, 98%, 99%, or 100%) identical to a human antibody constant region, such as an human IgG1 constant region, a human IgG2 constant region, a human IgG3 constant region, or a human IgG4 constant region. In one embodiment, the antibody Fe domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to wild-type human IgG1 Fc sequence: DKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIE KTISKAKGQPREPQVYTLPPSRDELTKNQVSLTCLVKGFYPSDIAVEWESNGQPENN YKTTPPVLDSDGSFFLYSKLTVDKSRWQQGNVFSCSVMHEALHNHYTQKSLSLSPG (SEQ ID NO:26). In some other embodiments, the amino acid sequence of the constant region is at least 90% (e.g., at least 91%, at least 92%, at least 93%, at least 94%, at least 95%, at least 96%, at least 97%, at least 98%, at least 99%, or 100%) identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, the antigen-binding site is linked to a portion of an antibody Fc domain sufficient to bind CD16. Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

In some embodiments, the antibody constant domain comprises a CH2 domain and a CH3 domain of an IgG antibody, for example, a human IgG1 antibody. In some embodiments, mutations are introduced in the antibody constant domain to enable heterodimerization with another antibody constant domain. For example, if the antibody constant domain is derived from the constant domain of a human IgG1, the antibody constant domain can comprise an amino acid sequence at least 90% (e.g., at least 91%, at least 92%, at least 93%, at least 94%, at least 95%, at least 96%, at least 97%, at least 98%, at least 99%, or 100%) identical to amino acids 234-332 of a human IgG1 antibody, and differs at one or more positions selected from the group consisting of Q347, Y349, L351, S354, E356, E357, K360, Q362, S364, T366, L368, K370, N390, K392, T394, D399, S400, D401, F405, Y407, K409, T411, and K439. All the amino acid positions in an Fc domain or hinge region disclosed herein are numbered according to EU numbering.

To facilitate formation of an asymmetric protein, Fc domain heterodimerization is contemplated. Mutations (e.g., amino acid substitutions) in the Fc domain that promote heterodimerization are described, for example, in International Application Publication No. WO2019157366, which is not incorporated herein by reference.

The proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the first immunoglobulin light chain can be cloned into a third expression vector; a fourth nucleic acid sequence encoding the second immunoglobulin light chain can be cloned into a fourth expression vector; the first, second, third and fourth expression vectors can be stably transfected together into host cells to produce the multimeric proteins.

To achieve the highest yield of the proteins, different ratios of the first, second, third and fourth expression vectors can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of a protein comprising an antigen-binding site disclosed herein. The protein can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

Accordingly, in another aspect, the present application provides one or more isolated nucleic acids comprising sequences encoding an immunoglobulin heavy chain and/or immunoglobulin light chain variable region of any one of the foregoing antibodies. The application provides one or more expression vectors that express the immunoglobulin heavy chain and/or immunoglobulin light chain variable region of any one of the foregoing antibodies. Similarly, the application provides host cells comprising one or more of the foregoing expression vectors and/or isolated nucleic acids.

Competition assays for determining whether an antibody binds to the same epitope as, or competes for binding with, a disclosed antibody are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen (e.g., a human EGFR protein or fragment thereof) bound to a solid surface or expressed on a cell surface, a test EGFR-binding antibody and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. For example, in certain embodiments, an optimized antibody has a higher affinity for the antigen when compared to the parental antibody.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

### CAR T Cells, EGFR/CD3-Directed Bispecific T-Cell Engagers, Immunocytokines, Antibody-Drug Conjugates, and Immunotoxins

Another aspect of the present application provides a molecule or complex comprising an antigen-binding site that binds EGFR as disclosed herein. Exemplary molecules or complexes include but are not limited to chimeric antigen receptors (CARs), T-cell engagers (e.g., EGFR/CD3-directed bispecific T-cell engagers), immunocytokines, antibody-drug conjugates, and immunotoxins.

Any antigen-binding site that binds EGFR as disclosed herein can be used. In certain embodiments, the VH, VL, and/or CDR sequences of the antigen-binding site that binds EGFR are provided in Table 1. In certain embodiments, the antigen-binding site that binds EGFR is an scFv. In certain embodiments, the scFv comprises an amino acid sequence at least 90% (e.g., at least 91%, at least 92%, at least 93%, at least 94%, at least 95%, at least 96%, at least 97%, at least 98%, or at least 99%) identical to an amino acid sequence selected from the group consisting of SEQ ID NOs: 14, 15, 18, 19, 20, 21, 24, and 25. In certain embodiments, the scFv comprises an amino acid sequence selected from the group consisting of SEQ ID NOs: 14, 15, 18, 19, 20, 21, 24, and 25.

In certain embodiments, the antigen-binding site that binds EGFR in the molecule or complex (e.g., CAR, T-cell engager, immunocytokine, antibody-drug conjugate, or immunotoxin) comprises S62R in the VH, F87Y in the VL, and D92R in the VL mutations relative to panitumumab, under the Chothia numbering scheme. In certain embodiments, the antigen-binding site that binds EGFR comprises a VH that comprises an amino acid sequence at least 90% (e.g., at least 91%, at least 92%, at least 93%, at least 94%, at least 95%, at least 96%, at least 97%, at least 98%, or at least 99%) identical to SEQ ID NO:1, and a VL that comprises an amino acid sequence at least 90% (e.g., at least 91%, at least 92%, at least 93%, at least 94%, at least 95%, at least 96%, at least 97%, at least 98%, or at least 99%) identical to SEQ ID NO:5. In certain embodiments, the antigen-binding site that binds EGFR in the molecule or complex (e.g., CAR, T-cell engager, immunocytokine, antibody-drug conjugate, or immunotoxin) comprises a heavy chain variable domain comprising CDR1, CDR2, and CDR3 sequences represented by the amino acid sequences of SEQ ID NOs: 2, 12, and 4, respectively; and a light chain variable domain comprising CDR1, CDR2, and CDR3 sequences represented by the amino acid sequences of SEQ ID NOs: 6, 7, and 17, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:11; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:16. In certain embodiments, the antigen-binding site comprises a heavy chain variable domain with an amino acid sequence comprising SEQ ID NO:32; and a light chain variable domain with an amino acid sequence comprising SEQ ID NO:33. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:18 or SEQ ID NO:19. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:18. In certain embodiments, the antigen-binding site comprises an scFv comprising an amino acid sequence identical to SEQ ID NO: 18 or 19. In certain embodiments, the antigen-binding site comprises an scFv comprising an amino acid sequence identical to SEQ ID NO:18. In certain embodiments, the antigen-binding site comprises an scFv comprising an amino acid sequence identical to SEQ ID NO:19.

In certain embodiments, the antigen-binding site that binds EGFR in the molecule or complex (e.g., CAR, T-cell engager, immunocytokine, antibody-drug conjugate, or immunotoxin) comprises F87Y and D92R mutations in the VL relative to panitumumab, under the Chothia numbering scheme. In certain embodiments, the antigen-binding site that binds EGFR comprises a VH that comprises an amino acid sequence at least 90% (e.g., at least 91%, at least 92%, at least 93%, at least 94%, at least 95%, at least 96%, at least 97%, at least 98%, or at least 99%) identical to SEQ ID NO:1, and a VL that comprises an amino acid sequence at least 90% (e.g., at least 91%, at least 92%, at least 93%, at least 94%, at least 95%, at least 96%, at least 97%, at least 98%, or at least 99%) identical to SEQ ID NO:5. In certain embodiments, the antigen-binding site that binds EGFR in the molecule or complex (e.g., CAR, T-cell engager, immunocytokine, antibody-drug conjugate, or immunotoxin) comprises a heavy chain variable domain comprising CDR1, CDR2, and CDR3 sequences represented by the amino acid sequences of SEQ ID NOs: 2, 3, and 4, respectively; and a light chain variable domain comprising CDR1, CDR2, and CDR3 sequences represented by the amino acid sequences of SEQ ID NOs: 6, 7, and 17, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:1; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:16. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:20 or SEQ ID NO:21. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:20. In certain embodiments, the antigen-binding site comprises an scFv comprising an amino acid sequence identical to SEQ ID NO: 20 or 21. In certain embodiments, the antigen-binding site comprises an scFv comprising an amino acid sequence identical to SEQ ID NO:20. In certain embodiments, the antigen-binding site comprises an scFv comprising an amino acid sequence identical to SEQ ID NO:21.

In certain embodiments, the antigen-binding site that binds EGFR in the molecule or complex (e.g., CAR, T-cell engager, immunocytokine, antibody-drug conjugate, or immunotoxin) comprises S62R in the VH and F87Y in the VL mutations relative to panitumumab, under the Chothia numbering scheme. In certain embodiments, the antigen-binding site that binds EGFR comprises a VH that comprises an amino acid sequence at least 90% (e.g., at least 91%, at least 92%, at least 93%, at least 94%, at least 95%, at least 96%, at least 97%, at least 98%, or at least 99%) identical to SEQ ID NO:1, and a VL that comprises an amino acid sequence at least 90% (e.g., at least 91%, at least 92%, at least 93%, at least 94%, at least 95%, at least 96%, at least 97%, at least 98%, or at least 99%) identical to SEQ ID NO:5. In certain embodiments, the antigen-binding site that binds EGFR in the molecule or complex (e.g., CAR, T-cell engager, immunocytokine, antibody-drug conjugate, or immunotoxin) comprises a heavy chain variable domain comprising CDR1, CDR2, and CDR3 sequences represented by the amino acid sequences of SEQ ID NOs: 2, 12, and 4, respectively; and a light chain variable domain comprising CDR1, CDR2, and CDR3 sequences represented by the amino acid sequences of SEQ ID NOs: 6, 7, and 8, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:11; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:13. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:14 or SEQ ID NO:15. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:14. In certain embodiments, the antigen-binding site comprises an scFv comprising an amino acid sequence identical to SEQ ID NO: 14 or 15. In certain embodiments, the antigen-binding site comprises an scFv comprising an amino acid sequence identical to SEQ ID NO:14. In certain embodiments, the antigen-binding site comprises an scFv comprising an amino acid sequence identical to SEQ ID NO:15.

In certain embodiments, the antigen-binding site that binds EGFR in the molecule or complex (e.g., CAR, T-cell engager, immunocytokine, antibody-drug conjugate, or immunotoxin) comprises F87Y in the VL, D92R in the VL, and optionally S62R in the VH mutations relative to panitumumab, under the Chothia numbering scheme. In certain embodiments, the antigen-binding site that binds EGFR comprises a VH that comprises an amino acid sequence at least 90% (e.g., at least 91%, at least 92%, at least 93%, at least 94%, at least 95%, at least 96%, at least 97%, at least 98%, or at least 99%) identical to SEQ ID NO:1, and a VL that comprises an amino acid sequence at least 90% (e.g., at least 91%, at least 92%, at least 93%, at least 94%, at least 95%, at least 96%, at least 97%, at least 98%, or at least 99%) identical to SEQ ID NO:5. In certain embodiments, the antigen-binding site that binds EGFR in the molecule or complex (e.g., CAR, T-cell engager, immunocytokine, antibody-drug conjugate, or immunotoxin) comprises a heavy chain variable domain comprising CDR1, CDR2, and CDR3 sequences represented by the amino acid sequences of SEQ ID NOs: 2, 23, and 4, respectively; and a light chain variable domain comprising CDR1, CDR2, and CDR3 sequences represented by the amino acid sequences of SEQ ID NOs: 6, 7, and 17, respectively. In certain embodiments, the antigen-binding site comprises a heavy chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:22; and a light chain variable domain with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:16. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:24 or SEQ ID NO:25. In certain embodiments, the antigen-binding site comprises an scFv comprising an amino acid sequence at least 90% (e.g., at least 91%, at least 92%, at least 93%, at least 94%, at least 95%, at least 96%, at least 97%, at least 98%, at least 99%, or 100%) identical to SEQ ID NO:24. In certain embodiments, the antigen-binding site comprises an scFv comprising an amino acid sequence identical to SEQ ID NO: 24 or 25. In certain embodiments, the antigen-binding site comprises an scFv comprising an amino acid sequence identical to SEQ ID NO:24. In certain embodiments, the antigen-binding site comprises an scFv comprising an amino acid sequence identical to SEQ ID NO:25.

### Chimeric Antigen Receptors (CARs)

In certain embodiments, the present application provides an EGFR-targeting CAR comprising an antigen-binding site that binds EGFR as disclosed herein (see, e.g., Table 1). The EGFR-targeting CAR can comprise a Fab fragment or an scFv. In certain embodiments, the antigen-binding site that binds EGFR in the CAR comprises one or more mutations selected from S62R in the VH, F87Y in the VL, and D92R in the VL relative to panitumumab, under the Chothia numbering scheme. In certain embodiments, the antigen-binding site that binds EGFR in the CAR comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from the group consisting of SEQ ID NOs: 14, 15, 18, 19, 20, 21, 24, and 25.

The term “chimeric antigen receptor” or alternatively a “CAR” refers to a recombinant polypeptide construct comprising at least an extracellular antigen binding domain, a transmembrane domain and an intracellular signaling domain comprising a functional signaling domain derived from a stimulatory molecule (also referred to herein as a “primary signaling domain”).

Accordingly, in certain embodiments, the CAR comprises an extracellular antigen-binding site that binds EGFR as disclosed herein, a transmembrane domain, and an intracellular signaling domain comprising a primary signaling domain. In certain embodiments, the CAR further comprises one or more functional signaling domains derived from at least one costimulatory molecule (also referred to as a “costimulatory signaling domain”).

In certain embodiments, the CAR comprises a chimeric fusion protein comprising an antigen-binding site that binds EGFR (e.g., EGFR-binding scFv) disclosed herein as an extracellular antigen binding domain, a transmembrane domain, and an intracellular signaling domain comprising a primary signaling domain. In certain embodiments, the CAR comprises a chimeric fusion protein comprising an antigen-binding site that binds EGFR (e.g., EGFR-binding scFv) disclosed herein as an extracellular antigen binding domain, a transmembrane domain, and an intracellular signaling domain comprising a costimulatory signaling domain and a primary signaling domain. In certain embodiments, the CAR comprises a chimeric fusion protein comprising an antigen-binding site that binds EGFR (e.g., EGFR-binding scFv) disclosed herein as an extracellular antigen binding domain, a transmembrane domain, and an intracellular signaling domain comprising two costimulatory signaling domains and a primary signaling domain. In certain embodiments, the CAR comprises a chimeric fusion protein comprising an antigen-binding site that binds EGFR (e.g., EGFR-binding scFv) disclosed herein as an extracellular antigen binding domain, a transmembrane domain, and an intracellular signaling domain comprising at least two costimulatory signaling domains and a primary signaling domain.

With respect to the transmembrane domain, in various embodiments, the CAR is designed to comprise a transmembrane domain that is fused to the extracellular domain of the CAR. In one embodiment, the transmembrane domain is one that naturally is associated with one of the domains in the CAR. In some instances, the transmembrane domain can be selected or modified by amino acid substitution to avoid binding of such domains to the transmembrane domains of the same or different surface membrane proteins to minimize interactions with other members of the receptor complex. In another embodiment, the transmembrane domain is capable of homodimerization with another CAR on the CAR T cell surface. In another embodiment, the amino acid sequence of the transmembrane domain may be modified or substituted so as to minimize interactions with the binding domains of the native binding partner present in the same CAR T cell.

The transmembrane domain may be derived from any naturally occurring membrane-bound or transmembrane protein. In one embodiment, the transmembrane region is capable of signaling to the intracellular domain(s) whenever the CAR has bound to a target. In some embodiments, the transmembrane domain comprises the transmembrane region(s) of one or more proteins selected from the group consisting of TCR α chain, TCR β chain, TCR ζ chain, CD28, CD3F, CD45, CD4, CD5, CD8, CD9, CD16, CD22, EGFR, CD37, CD64, CD80, CD86, CD134, CD137, and CD154. In some embodiments, the transmembrane domain comprises the transmembrane region(s) of one or more proteins selected from the group consisting of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C.

The extracellular EGFR-binding domain (e.g., EGFR-binding scFv domain) domain can be connected to the transmembrane domain by a hinge region. A variety of hinges can be employed, including but not limited to the human Ig (immunoglobulin) hinge (e.g., an IgG4 hinge, an IgD hinge), a Gly-Ser linker, a (G₄S)₄ linker (SEQ ID NO:31), a KIR2DS2 hinge, and a CD8α hinge.

The intracellular signaling domain of the CAR described in the present application is responsible for activation of at least one of the specialized functions of the immune cell (e.g., cytolytic activity or helper activity, including the secretion of cytokines, of a T cell) in which the CAR has been placed in. Thus, as used herein, the term “intracellular signaling domain” refers to the portion of a protein which transduces an effector function signal and directs the cell to perform a specialized function. Although usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain of the CAR comprises a primary signaling domain (i.e., a functional signaling domain derived from a stimulatory molecule) and one or more costimulatory signaling domains (i.e., functional signaling domains derived from at least one costimulatory molecule).

As used herein, the term “stimulatory molecule” refers to a molecule expressed by an immune cell, e.g., a T cell, an NK cell, or a B cell, that provide the cytoplasmic signaling sequence(s) that regulate activation of the immune cell in a stimulatory way for at least some aspect of the immune cell signaling pathway. In one embodiment, the signal is a primary signal that is initiated by, for instance, binding of a TCR/CD3 complex with an MHC molecule loaded with a peptide, and which leads to mediation of a T cell response, including, but not limited to, proliferation, activation, differentiation, and the like.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the present application include those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In one embodiment, the primary signaling domain in any one or more CARs described in the present application comprises a cytoplasmic signaling sequence derived from CD3-zeta.

In some embodiments, the primary signaling domain is a functional signaling domain of TCR zeta, FcR gamma, FcR beta, CD3 gamma, CD3 delta, CD3 epsilon, CD5, CD22, CD79a, CD79b, CD66d, 4-1BB, and/or CD3-zeta. In an embodiment, the intracellular signaling domain comprises a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and/or DAP12. In a particular embodiment, the primary signaling domain is a functional signaling domain of the zeta chain associated with the T cell receptor complex.

As used herein, the term “costimulatory molecule” refers to a cognate binding partner on a T cell that specifically binds with a costimulatory ligand, thereby mediating a costimulatory response by the T cell, such as, but not limited to, proliferation. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1, CD11a/CD18), CD2, CD7, CD258 (LIGHT), NKG2C, B7-H3, and a ligand that specifically binds with CD83, and the like. Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and a ligand that specifically binds with CD83. In some embodiments, the costimulatory signaling domain of the CAR is a functional signaling domain of a costimulatory molecule described herein, e.g., OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

As used herein, the term “signaling domain” refers to the functional portion of a protein which acts by transmitting information within the cell to regulate cellular activity via defined signaling pathways by generating second messengers or functioning as effectors by responding to such messengers.

The cytoplasmic signaling sequences within the cytoplasmic signaling portion of the CAR described in the present application may be linked to each other in a random or specified order. Optionally, a short oligo- or polypeptide linker, for example, between 2 and 10 amino acids in length may form the linkage.

Another aspect of the present application provides a nucleic acid encoding an EGFR-targeting CAR disclosed herein. The nucleic acid is useful for expressing the CAR in an effector cell (e.g., T cell) by introducing the nucleic acid to the cell.

Modifications may be made in the sequence to create an equivalent or improved variant, for example, by changing one or more of the codons according to the codon degeneracy table. A DNA codon degeneracy table is provided in Table 3.

TABLE 3
Amino Acid Codons

| | One | Three | |
|---|---|---|---|
| | letter | letter | |
| Amino Acids | code | code | Codons |
| Alanine | A | Ala | GCA GCC GCG GCU |
| Cysteine | C | Cys | UGC UGU |
| Aspartic acid | D | Asp | GAC GAU |
| Glutamic acid | E | Glu | GAA GAG |
| Phenylalanine | F | Phe | UUC UUU |
| Glycine | G | Gly | GGA GGC GGG GGU |
| Histidine | H | His | CAC CAU |
| Isoleucine | I | Iso | AUA AUC AUU |
| Lysine | K | Lys | AAA AAG |
| Leucine | L | Leu | UUA UUG CUA CUC CUG CUU |
| Methionine | M | Met | AUG |
| Asparagine | N | Asn | AAC AAU |
| Proline | P | Pro | CCA CCC CCG CCU |
| Glutamine | Q | Gln | CAA CAG |
| Arginine | R | Arg | AGA AGG CGA CGC CGG CGU |
| Serine | S | Ser | AGC AGU UCA UCC UCG UCU |
| Threonine | T | Thr | ACA ACC ACG ACU |
| Valine | V | Val | GUA GUC GUG GUU |
| Tryptophan | W | Trp | UGG |
| Tyrosine | Y | Tyr | UAC UAU |

In certain embodiments, the nucleic acid is a DNA molecule (e.g., a cDNA molecule). In certain embodiments, the nucleic acid further comprises an expression control sequence (e.g., promoter and/or enhancer) operably linked to the CAR coding sequence. In certain embodiments, the present application provides a vector comprising the nucleic acid. The vector can be a viral vector (e.g., AAV vector, lentiviral vector, or adenoviral vector) or a non-viral vector (e.g., plasmid).

In certain embodiments, the nucleic acid is an RNA molecule (e.g., an mRNA molecule). A method for generating mRNA for use in transfection can involve in vitro transcription of a template with specially designed primers, followed by polyA addition, to produce an RNA construct containing 3′ and 5′ untranslated sequences, a 5′ cap and/or Internal Ribosome Entry Site (IRES), the nucleic acid to be expressed, and a polyA tail, typically 50-2000 bases in length. The RNA molecule can be further modified to increase translational efficiency and/or stability, e.g., as disclosed in U.S. Pat. Nos. 8,278,036; 8,883,506, and 8,716,465. RNA molecules so produced can efficiently transfect different kinds of cells.

In one embodiment, the nucleic acid encodes an amino acid sequence comprising a signal peptide at the amino-terminus of the CAR. Such signal peptide can facilitate the cell surface localization of the CAR when it is expressed in an effector cell, and is cleaved from the CAR during cellular processing. In one embodiment, the nucleic acid encodes an amino acid sequence comprising a signal peptide at the N-terminus of the extracellular EGFR-binding domain (e.g., EGFR-binding scFv domain).

RNA or DNA can be introduced into target cells using any of a number of different methods, for instance, commercially available methods which include, but are not limited to, electroporation, cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)).

In another aspect, the present application provides an immune effector cell expressing the EGFR-targeting CAR. Also provided is an immune effector cell comprising the nucleic acid encoding the EGFR-targeting CAR. The immune effector cells include but are not limited to T cells and NK cells. In certain embodiments, the T cell is selected from a CD8⁺ T cell, a CD4⁺ T cell, a γδ T cell, and an NKT cell. The T cell or NK cell can be a primary cell or a cell line.

The immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors, by methods known in the art. The immune effector cells can also be differentiated in vitro from a pluripotent or multipotent cell (e.g., a hematopoietic stem cell). In some embodiments, the present application provides a pluripotent or multipotent cell (e.g., a hematopoietic stem cell) expressing the EGFR-targeting CAR (e.g., expressing the CAR on the plasma membrane) or comprising a nucleic acid disclosed herein.

In certain embodiments, the immune effector cells are isolated and/or purified. For example, regulatory T cells can be removed from a T cell population using a CD25-binding ligand. Effector cells expressing a checkpoint protein (e.g., PD-1, LAG-3, or TIM-3) can be removed by similar methods. In certain embodiments, the effector cells are isolated by a positive selection step. For example, a population of T cells can be isolated by incubation with anti-CD3/anti-CD28-conjugated beads. Other cell surface markers, such as IFN-7, TNF-α, IL-17A, IL-2, IL-3, IL-4, GM-CSF, IL-10, IL-13, granzyme B, and perforin, can also be used for positive selection.

Immune effector cells may be activated and expanded generally using methods known in the art, e.g., as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publications Nos. 2006/0121005 and 2016/0340406. For example, in certain embodiments, T cells can be expanded and/or activated by contact with an anti-CD3 antibody and an anti-CD28 antibody, under conditions appropriate for stimulating proliferation of the T cells. The cells can be expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. Multiple cycles of stimulation may be desirable for prolonged cell culture (e.g., culture for a period of 60 days or more). In certain embodiments, the cell culture comprises serum (e.g., fetal bovine or human serum), interleukin-2 (IL-2), insulin, IFN-7, IL-4, IL-7, GM-CSF, IL-10, IL-12, IL-15, TGFβ, TNF-α, or a combination thereof. Other additives for the growth of cells known to the skilled person, e.g., surfactant, plasmanate, and reducing agents such as N-acetyl-cysteine and 2-mercaptoethanol, can also be included in the cell culture. In certain embodiments, the immune effector cell of the present application is a cell obtained from in vitro expansion.

Further designs of CAR constructs that may comprise an antigen-binding site that binds EGFR (e.g., regulatable CAR), nucleic acid encoding the CAR, and effector cells expressing the CAR or comprising the nucleic acid are provided in U.S. Pat. Nos. 7,446,190 and 9,181,527, U.S. Patent Application Publication Nos. 2016/0340406 and 2017/0049819, and International Patent Application Publication No. WO2018/140725.

### EGFR CD3-Directed Bispecific T-Cell Engagers

In certain embodiments, the present application provides an EGFR/CD3-directed bispecific T-cell engager comprising an antigen-binding site that binds EGFR disclosed herein. In certain embodiments, the antigen-binding site that binds EGFR in the EGFR/CD3-directed bispecific T-cell engager comprises one or more mutations selected from S62R in the VH, F87Y in the VL, and D92R in the VL relative to panitumumab, under the Chothia numbering scheme. In certain embodiments, the EGFR/CD3-directed bispecific T-cell engager comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from the group consisting of SEQ ID NOs: 14, 15, 18, 19, 20, 21, 24, and 25. In certain embodiments, the cytokine is connected to the Fc domain directly or via a linker.

In certain embodiments, the EGFR/CD3-directed bispecific T-cell engager further comprises an antigen-binding site that binds CD3. Exemplary antigen-binding sites that bind CD3 are disclosed in International Patent Application Publication Nos. WO2014/051433 and WO2017/097723.

Another aspect of the present application provides a nucleic acid encoding at least one polypeptide of the EGFR/CD3-directed bispecific T-cell engager, wherein the polypeptide comprises an antigen-binding site that binds EGFR. In certain embodiments, the nucleic acid further comprises a nucleotide sequence encoding a signal peptide that, when expressed, is at the N-terminus of one or more of the polypeptides of the EGFR/CD3-directed bispecific T-cell engager. Also provided is a vector (e.g., a viral vector) comprising the nucleic acid, a producer cell comprising the nucleic acid or vector, and a producer cell expressing the EGFR/CD3-directed bispecific T-cell engager.

### Immunocytokines

In certain embodiments, the present application provides an immunocytokine comprising an antigen-binding site that binds EGFR disclosed herein and a cytokine. Any cytokine (e.g., pro-inflammatory or anti-inflammatory cytokines) known in the art can be used, including but not limited to IL-2, IL-4, IL-10, IL-12, IL-15, TNFα, IFNα, IFNγ, and GM-CSF. More exemplary cytokines are disclosed in U.S. Pat. No. 9,567,399. In certain embodiments, the antigen-binding site is connected to the cytokine by chemical conjugation (e.g., covalent or noncovalent chemical conjugation). In certain embodiments, the antigen-binding site is connected to the cytokine by fusion of the polypeptide chains (i.e., peptide linkage). The immunocytokine can further comprise an Fc domain connected to the antigen-binding site that binds EGFR. In certain embodiments, the antigen-binding site that binds EGFR in the immunocytokine comprises one or more mutations selected from S62R in the VH, F87Y in the VL, and D92R in the VL relative to panitumumab, under the Chothia numbering scheme. In certain embodiments, the immunocytokine comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from the group consisting of SEQ ID NOs: 14, 15, 18, 19, 20, 21, 24, and 25. In certain embodiments, the cytokine is connected to the Fc domain directly or via a linker.

In another aspect, the present application provides a nucleic acid encoding at least one polypeptide of the immunocytokine, wherein the polypeptide comprises an antigen-binding site that binds EGFR. In certain embodiments, the nucleic acid further comprises a nucleotide sequence encoding a signal peptide that, when expressed, is at the N-terminus of one or more of the polypeptides of the immunocytokine. Also provided is a vector (e.g., a viral vector) comprising the nucleic acid, a producer cell comprising the nucleic acid or vector, and a producer cell expressing the immunocytokine.

### Antibody-Drug Conjugates

In certain embodiments, the present application provides an antibody-drug conjugate comprising an antigen-binding site that binds EGFR disclosed herein and a cytotoxic drug moiety. Exemplary cytotoxic drug moieties are disclosed in International Patent Application Publication Nos. WO2014/160160 and WO2015/143382. In certain embodiments, the cytotoxic drug moiety is selected from auristatin, N-acetyl-γ calicheamicin, maytansinoid, pyrrolobenzodiazepine, and SN-38. The antigen-binding site can be connected to the cytotoxic drug moiety by chemical conjugation (e.g., covalent or noncovalent chemical conjugation). In certain embodiments, the antibody-drug conjugate further comprises an Fc domain connected to the antigen-binding site that binds EGFR. In certain embodiments, the antigen-binding site that binds EGFR in the antibody-drug conjugate comprises one or more mutations selected from S62R in the VH, F87Y in the VL, and D92R in the VL relative to panitumumab, under the Chothia numbering scheme. In certain embodiments, the antibody-drug conjugate comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from the group consisting of SEQ ID NOs: 14, 15, 18, 19, 20, 21, 24, and 25. In certain embodiments, the cytotoxic drug moiety is connected to the Fc domain directly or via a linker.

### Immunotoxins

In certain embodiments, the present application provides an immunotoxin comprising an antigen-binding site that binds EGFR disclosed herein and a cytotoxic peptide moiety. Any cytotoxic peptide moiety known in the art can be used, including but not limited to ricin, Diphtheria toxin, and Pseudomonas exotoxin A. More exemplary cytotoxic peptides are disclosed in International Patent Application Publication Nos. WO2012/154530 and WO2014/164680. In certain embodiments, the cytotoxic peptide moiety is connected to the protein by chemical conjugation (e.g., covalent or noncovalent chemical conjugation). In certain embodiments, the cytotoxic peptide moiety is connected to the protein by fusion of polypeptide. The immunotoxin can further comprise an Fc domain connected to the antigen-binding site that binds EGFR. In certain embodiments, the antigen-binding site that binds EGFR in the immunotoxin comprises one or more mutations selected from S62R in the VH, F87Y in the VL, and D92R in the VL relative to panitumumab, under the Chothia numbering scheme. In certain embodiments, the immunotoxin comprises an amino acid sequence at least 90% (e.g., at least 91%, at least 92%, at least 93%, at least 94%, at least 95%, at least 96%, at least 97%, at least 98%, at least 99%, or 100%) identical to an amino acid sequence selected from the group consisting of SEQ ID NOs: 14, 15, 18, 19, 20, 21, 24, and 25. In certain embodiments, the cytotoxic peptide moiety is connected to the Fc domain directly or via a linker.

In another aspect, the present application provides a nucleic acid encoding at least one polypeptide of the immunotoxin, wherein the polypeptide comprises an antigen-binding site that binds EGFR. In certain embodiments, the nucleic acid further comprises a nucleotide sequence encoding a signal peptide that, when expressed, is at the N-terminus of one or more of the polypeptides of the immunotoxin. Also provided is a vector (e.g., a viral vector) comprising the nucleic acid, a producer cell comprising the nucleic acid or vector, and a producer cell expressing the immunotoxin.

II. Therapeutic Compositions and their Use

The present application provides methods for treating cancer using a protein, conjugate, or cells comprising an antigen-binding site disclosed herein and/or a pharmaceutical composition described herein. The methods may be used to treat a variety of cancers which express EGFR by administering to a patient in need thereof a therapeutically effective amount of a protein, conjugate, or cells comprising an antigen-binding site disclosed herein.

The therapeutic method can be characterized according to the cancer to be treated. The cancer to be treated can be characterized according to the presence of a particular antigen expressed on the surface of the cancer cell.

Cancers characterized by the expression of EGFR, include, without limitation, solid tumor cancers. For example, in certain embodiments, the cancer is head and neck cancer, colorectal cancer, non-small cell lung cancer, glioma, renal cell carcinoma, bladder cancer, cervical cancer, ovarian cancer, pancreatic cancer, or liver cancer. In certain embodiments, the cancer is lung cancer (including, but not limited to, small cell lung carcinoma or lung adenocarcinoma), breast cancer, breast invasive ductal carcinoma, kidney cancer, conventional glioblastoma multiforme, colon cancer, colon adenocarcinoma, gastric cancer, brain cancer, glioblastoma, bladder, head and neck cancers, ovarian cancer or prostate cancer.

It is contemplated that the protein, conjugate, cells, and/or the pharmaceutical compositions described herein can be used to treat a variety of cancers, not limited to cancers in which the cancer cells or the cells in the cancer microenvironment express EGFR.

In certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The cancer to be treated can be characterized according to the presence of a particular antigen expressed on the surface of the cancer cell. In certain embodiments, the cancer cell can express one or more of the following in addition to EGFR: CD2, CD19, CD38, CD40, CD52, CD30, CD70, IGF1R, HER3/ERBB3, HER4/ERBB4, MUC1, TROP2, cMET, SLAMF7, PSCA, MICA, MICB, TRAILR1, TRAILR2, MAGE-A3, B7.1, B7.2, CTLA4, and PD1.

### II. Combination Therapy

In another aspect, the present application provides for combination therapy. Proteins, conjugates, and cells comprising an antigen-binding site described herein can be used in combination with additional therapeutic agents to treat the cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK inhibitor, an ATR inhibitor, an A2A antagonist, a base excision repair inhibitor, a Bcr-Abl tyrosine kinase inhibitor, a Bruton's tyrosine kinase inhibitor, a CDC7 inhibitor, a CHK1 inhibitor, a Cyclin-Dependent Kinase inhibitor, a DNA-PK inhibitor, an inhibitor of both DNA-PK and mTOR, a DNMT1 inhibitor, a DNMT1 inhibitor plus 2-chloro-deoxyadenosine, an HDAC inhibitor, a Hedgehog signaling pathway inhibitor, an IDO inhibitor, a JAK inhibitor, an mTOR inhibitor, a MEK inhibitor, a MELK inhibitor, a MTH1 inhibitor, a PARP inhibitor, a Phosphoinositide 3-Kinase inhibitor, an inhibitor of both PARP1 and DHODH, a proteasome inhibitor, a Topoisomerase-II inhibitor, a tyrosine kinase inhibitor, a VEGFR inhibitor, and a WEE1 inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins described in the present application can also be used as an adjunct to surgical removal of the primary lesion.

The amount of the protein, conjugate, or cells disclosed herein and the additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a protein, conjugate, or cell disclosed herein may be administered during a time when the additional therapeutic agent(s) exerts its prophylactic or therapeutic effect, or vice versa.

### IV. Pharmaceutical Compositions

The present disclosure also features pharmaceutical compositions that contain a therapeutically effective amount of a protein, protein conjugate, or immune effector cell described herein. The composition can be formulated for use in a variety of drug delivery systems. One or more physiologically acceptable excipients or carriers can also be included in the composition for proper formulation. Suitable formulations for use in compositions disclosed herein are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In one aspect, the present disclosure provides a formulation of a protein, which contains an EGFR-binding site described herein, and a pharmaceutically acceptable carrier.

The composition can be formulated for use in a variety of drug delivery systems. One or more physiologically acceptable excipients or carriers can be included in the composition for proper formulation. Suitable formulations for use in the compositions disclosed herein are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In certain embodiments, the composition may be a drug delivery formulation. The intravenous drug delivery formulation described herein may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained in about 12-60 vials. In certain embodiments, the formulation may be freeze-dried and 45 mg of the freeze-dried formulation may be contained in one vial. In certain embodiments, the about 40 mg to about 100 mg of freeze-dried formulation may be contained in one vial. In certain embodiments, freeze-dried formulation from 12, 27, or 45 vials are combined to obtain a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, for example, between 5 and 9 or between 6 and 8, and in certain embodiments between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein described in the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including a protein described in the present disclosure in a pH-buffered solution. The buffer of the present application may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86 mg/mL), and about 6.2 mg/mL of sodium chloride (e.g., 6.165 mg/mL). In certain embodiments, the buffer system includes about 1 to about 1.5 mg/mL of citric acid, about 0.25 to about 0.5 mg/mL of sodium citrate, about 1.25 to about 1.75 mg/mL of disodium phosphate dihydrate, about 0.7 to about 1.1 mg/mL of sodium dihydrogen phosphate dihydrate, and about 6.0 to about 6.4 mg/mL of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to about 15 mg/mL. In certain embodiments, the concentration of mannitol may be about 10 to about 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product described herein is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the protein product solution in order to allow an extractable volume of 60 mL. In certain embodiments, the liquid formulation may be diluted with 0.9% saline solution.

In certain embodiments, the liquid formulation disclosed herein may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as a 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation described herein may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) delivery may be an administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

The protein described herein could exist in a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein described herein may be adjusted between 6 to 8. In certain embodiments, the pH range for the lyophilized drug product may be from 7 to 8.

In certain embodiments, a salt or buffer component may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present application may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be reconstituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative aqueous carriers include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product disclosed herein is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product disclosed herein is reconstituted to about 4.5 mL water for injection and diluted with 0.9% saline solution (sodium chloride solution).

Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 g to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 g to about 1 mg/kg of body weight, about 10 μg to about 100 μg/kg of body weight, about g to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the compositions described herein could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of a protein comprising an antigen-binding site described in the present application. The patent application specifically contemplates all combinations and permutations of the aspects and embodiments.

Throughout the description, where compositions are described as having, including, or comprising specific components, or where processes and methods are described as having, including, or comprising specific steps, it is contemplated that, additionally, there are compositions of a protein comprising an antigen-binding site described in the present application that consist essentially of, or consist of, the recited components, and that there are processes and methods according to the present application that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present application, whether explicit or implicit herein. For example, where reference is made to a particular compound, that compound can be used in various embodiments of compositions of a protein comprising an antigen-binding site described in the present application and/or in methods of a protein comprising an antigen-binding site described in the present application, unless otherwise understood from the context. In other words, within this application, embodiments have been described and depicted in a way that enables a clear and concise application to be written and drawn, but it is intended and will be appreciated that embodiments may be variously combined or separated without parting from the present teachings. For example, it will be appreciated that all features described and depicted herein can be applicable to all aspects of a protein comprising an antigen-binding site described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present application also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as a protein comprising an antigen-binding site described in the present application remain operable. Moreover, two or more steps or actions may be conducted simultaneously.

The use of any and all examples, or exemplary language herein, for example, “such as” or “including,” is intended merely to illustrate better a protein comprising an antigen-binding site described in the present application, and does not pose a limitation on the scope of a protein comprising an antigen-binding site described in the application unless claimed. No language in the specification should be construed as indicating any non-claimed element as essential to the practice of the present application.

### EXAMPLES

The following examples are merely illustrative and are not intended to limit the scope or content of the application in any way.

### Example 1. Selection of EGFR-Binding Interface Using SPR and DSC Analysis

This example was designed to develop EGFR-binding sites derived from panitumumab that have improved thermostability than panitumumab. Briefly, 25 constructs, each containing a single point mutation selected based on the crystal structure of panitumumab Fab in complex with the D3 domain of EGFR (PDB ID: 5SX4), were designed. Other than three constructs that did not express well, the EGFR-binding sites were produced to evaluate the mutation's impact on binding affinity and thermostability. Each scFv construct also contained G44C substitution in the VH and G100C substitution in the VL. Kinetics and affinity for human and Rhesus macaque EGFR was evaluated using surface plasmon resonance (SPR). The results are shown in Table 4. Given that the impact for each mutation was minute, those that caused marked reduction in affinity were removed from consideration. Thermostability of eight constructs was further assessed by Differential Scanning Fluorimetry (DSF) using the method described in Example 3. The results are shown in Table 5.

TABLE 4
SPR analysis of the 22 anti-EGFR constructs with single point mutations.

| Expected | | Human | Rhesus | Human | Rhesus | Human | Rhesus |
|---|---|---|---|---|---|---|---|
| Function | | EGFR | EGFR | EGFR | EGFR | EGFR | EGFR |
| of Design | Name* | kₐ (1/Ms) | kₐ (1/Ms) | k_{d} (1/s) | k_{d} (1/s) | K_{D} (M) | K_{D} (M) |
| 2nd pair of | PANI- | No binding | | | | | |
| disulfide | G106C- | | | | | | |
| bond | A43C | | | | | | |
| Stability | PANI-H- | 5.12E+05 | 4.36E+05 | 1.75E−03 | 1.78E−03 | 3.41E−09 | 4.08E−09 |
| | S62R | | | | | | |
| Stability | PANI-H- | 5.11E+05 | 4.35E+05 | 1.29E−03 | 1.34E−03 | 2.52E−09 | 3.08E−09 |
| | S62K | | | | | | |
| Stability | PANI-H- | 1.14E+05 | 1.17E+05 | 4.37E−03 | 5.37E−03 | 3.85E−08 | 4.60E−08 |
| | D101T | | | | | | |
| Stability | PANI-H- | 2.86E+05 | 3.49E+05 | 3.37E−03 | 4.73E−03 | 1.18E−08 | 1.35E−08 |
| | D101S | | | | | | |
| Stability | PANI-H- | No binding | | | | | |
| | D101N | | | | | | |
| Stability | PANI-H- | No binding | | | | | |
| | D101Q | | | | | | |
| Stability | PANI-L- | 5.22E+05 | 3.28E+05 | 8.74E−04 | 9.96E−04 | 1.68E−09 | 3.03E−09 |
| | F87Y | | | | | | |
| Stability | PANI-L- | 6.90E+05 | 5.05E+05 | 8.32E−03 | 8.98E−03 | 1.21E−08 | 1.78E−08 |
| | L96Q | | | | | | |
| Stability | PANI-L- | 5.34E+05 | 3.48E+05 | 3.24E−03 | 3.75E−03 | 6.07E−09 | 1.08E−08 |
| | L96N | | | | | | |
| Stability | PANI-L- | No binding | | | | | |
| | L96E | | | | | | |
| Stability | PANI-L- | 3.15E+05 | 2.54E+05 | 6.62E−03 | 8.08E−03 | 2.10E−08 | 3.18E−08 |
| | L96D | | | | | | |
| Affinity | PANI-H- | No | Not | No | Not | No | Not |
| | S54Q | binding | tested | binding | tested | binding | tested |
| Affinity | PANI-H- | 5.75E+05 | 4.22E+05 | 4.68E−03 | 5.65E−03 | 8.12E−09 | 1.34E−08 |
| | S54N | | | | | | |
| Affinity | PANI-H- | No binding | | | | | |
| | S54E | | | | | | |
| Affinity | PANI-H- | No binding | | | | | |
| | S54D | | | | | | |
| Affinity | PANI-H- | 3.90E+05 | 2.65E+05 | 5.84E−03 | 6.90E−03 | 1.50E−08 | 2.61E−08 |
| | T98Q | | | | | | |
| Affinity | PANI-H- | 7.46E+05 | 5.00E+05 | 4.21E−02 | 2.00E−02 | 5.64E−08 | 4.00E−08 |
| | T98N | | | | | | |
| Affinity | PANI-H- | 4.53E+05 | 3.77E+05 | 3.31E−03 | 3.53E−03 | 7.32E−09 | 9.35E−09 |
| | T98E | | | | | | |
| Affinity | PANI-H- | 6.07E+05 | 5.18E+05 | 8.30E−03 | 9.62E−03 | 1.37E−08 | 1.86E−08 |
| | T98D | | | | | | |
| Affinity/ | PANI-L- | 2.56E+05 | 1.89E+05 | 8.39E−04 | 9.60E−04 | 3.27E−09 | 5.09E−09 |
| Stability | D92R | | | | | | |
| Control | Original | 5.50E+05 | 3.67E+05 | 9.59E−04 | 1.08E−03 | 1.74E−09 | 2.95E−09 |
| | Pani-scFv | | | | | | |

*-H- denotes mutation in the heavy chain; -L- denotes mutation in the light chain; and PANI denotes the panitumumab framework.

TABLE 5
DSF analysis of 8 anti-EGFR constructs
with single point mutations.

| Expected Function | | | |
|---|---|---|---|
| of Design | Name* | Tₘ₁ (° C.) | Tₘ₂ (° C.) |
| Stability | PANI-H-S62R | 66.6 | 84.5 |
| Stability | PANI-L-F87Y | 66.0 | 84.3 |
| Stability | PANI-L-L96N | 62.4 | 84.1 |
| Affinity | PANI-H-S54Q | 65.4 | 86.3 |
| Affinity | PANI-H-S54N | 67.0 | 84.4 |
| Affinity | PANI-H-T98E | 62.4 | 83.0 |
| Affinity/Stability | PANI-L-D92R | 66.1 | 83.6 |
| Control | Original Pani-scFv | 66.0 | 84.0 |

*-H- denotes mutation in the heavy chain; -L- denotes mutation in the light chain; and PANI denotes the panitumumab framework.

Based on these results, the following mutations were determined to most effectively improve/retain affinity and thermostability:
Heavy Chain: S62R (under the Chothia numbering scheme)
Light Chain: F87Y, D92R (under the Chothia numbering scheme)

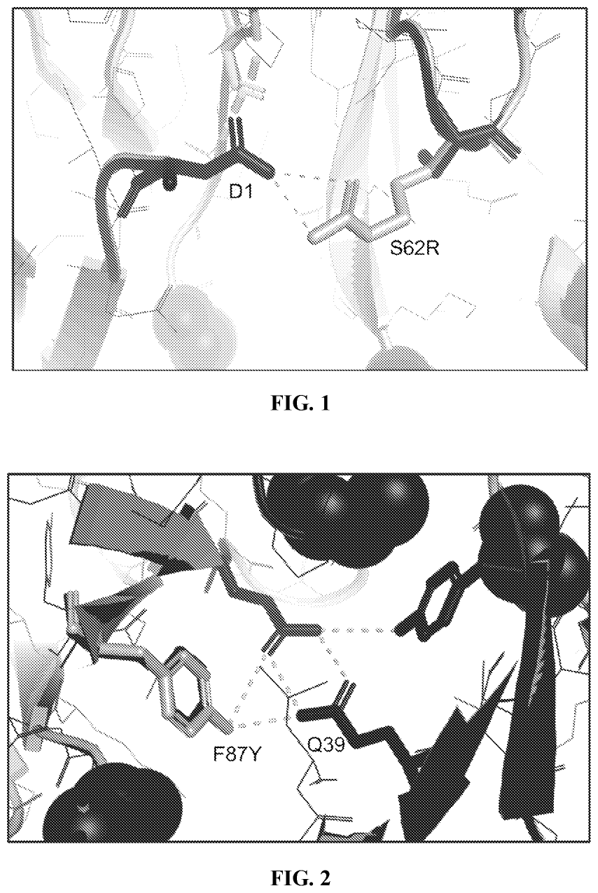

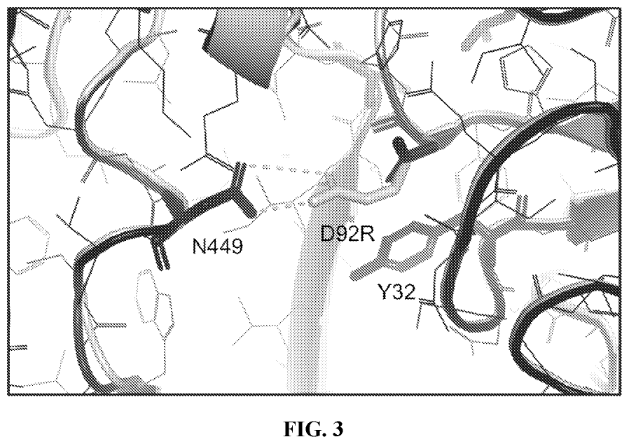

Structural modeling of these mutations are shown in FIGS. 1-3. As shown in FIG. 1, the S62R mutation of the VH chain of panitumumab was found to introduce additional hydrogen bonds with D1 of VL and contributes to the stability of the VH/VL interface according to the modeling. As shown in FIG. 2, the F87Y mutation of the VL chain of panitumumab was found to introduce additional hydrogen bonds with Q39 of VH and contributes to the stability of the VH/VL interface according to the modeling. As shown in FIG. 3, the D92R mutation at CDR3 of the VL chain of panitumumab, originally designed to improve affinity (for binding to N449 of EGFR), was found to unexpectedly form additional van der Waals' contacts with Y32 of CDRL1 and stabilizes the paratope, according to the modeling.

Thereafter, the impact of combinations of mutations on affinity and thermostability was evaluated using SPR and Differential Scanning Calorimetry (DSC), respectively. Each construct also contained a G44C substitution in the VH and a G100C substitution in the VL. The results from the SPR analysis are shown in Table 6 and the results from the DSC analysis are shown in Table 7.

TABLE 6
SPR analysis for combination mutations.

| Expected | | Human | Rhesus | Human | Rhesus | Human | Rhesus |
|---|---|---|---|---|---|---|---|
| Function | | EGFR | EGFR | EGFR | EGFR | EGFR | EGFR |
| of Design | Name* | kₐ (1/Ms) | | k_{d} (1/s) | | K_{D} (M) | |
| Stability | PANI- | 4.00E+05 | 2.79E+05 | 1.01E−03 | 1.16E−03 | 2.53E−09 | 4.15E−09 |
| Combo | H_S62R- | | | | | | |
| | L_F87Y | | | | | | |
| Stability/ | PANI- | 2.93E+05 | 2.09E+05 | 9.90E−04 | 1.12E−03 | 3.38E−09 | 5.36E−09 |
| Affinity | H_S62R- | | | | | | |
| Combo | L_F87Y- | | | | | | |
| | L_D92R | | | | | | |
| Stability/ | PANI- | No binding | | | | | |
| Affinity | H_S62R- | | | | | | |
| Combo | L_F87Y- | | | | | | |
| | H_T98R | | | | | | |
| Stability/ | PANI- | No binding | | | | | |
| Affinity | H_S62R- | | | | | | |
| Combo | L_F87Y- | | | | | | |
| | L_D92R- | | | | | | |
| | H_T98R | | | | | | |
| Stability/ | PANI- | 3.52E+05 | 2.43E+05 | 2.97E−03 | 3.30E−03 | 8.46E−09 | 1.35E−08 |
| Affinity | L_F87Y- | | | | | | |
| Combo | L_L96N- | | | | | | |
| | L_D92R | | | | | | |
| Stability/ | PANI- | Transient binding | | | | | |
| Affinity | L_F87Y- | | | | | | |
| Combo | L_L96N- | | | | | | |
| | H_T98R | | | | | | |
| Stability/ | PANI- | No expression | | | | | |
| Affinity | L_F87Y- | | | | | | |
| Combo | L_L96N- | | | | | | |
| | L_D92R- | | | | | | |
| | H_T98R | | | | | | |
| Stability/ | PANI- | 2.46E+05 | 1.82E+05 | 3.43E−03 | 3.89E−03 | 1.40E−08 | 2.14E−08 |
| Affinity | H_S62R- | | | | | | |
| Combo | L_F87Y- | | | | | | |
| | L_L96N- | | | | | | |
| | L_D92R | | | | | | |
| Stability/ | PANI- | No binding | | | | | |
| Affinity | H_S62R- | | | | | | |
| Combo | L_F87Y- | | | | | | |
| | L_L96N- | | | | | | |
| | H_T98R | | | | | | |
| Stability/ | PANI- | No binding | | | | | |
| Affinity | H_S62R- | | | | | | |
| Combo | L_F87Y- | | | | | | |
| | L_L96N- | | | | | | |
| | L_D92R- | | | | | | |
| | H_T98R | | | | | | |
| Stability/ | PANI- | 3.05E+05 | N/A | 7.94E−04 | N/A | 2.45E−09 | N/A |
| Affinity | L_F87Y- | | | | | | |
| Combo | L_D92R | | | | | | |
| Control | Original | 5.51E+05 | 3.77E+05 | 1.05E−03 | 1.20E−03 | 1.91E−09 | 3.17E−09 |
| | Pani-scFv | | | | | | |

*-H denotes mutation in the heavy chain; L- denotes mutation in the light chain; and PANI denotes the panitumumab framework.

TABLE 7
DSC analysis of 12 anti-EGFR constructs with single point mutations.

| Expected | | DSC | | | | |
|---|---|---|---|---|---|---|
| Function | | Tₒₙₛₑₜ | Tₘ₁ | Tₘ₂ | Tₘ₃ | Tₘ₄ |
| of Design | Name* | (° C.) | (° C.) | (° C.) | (° C.) | (° C.) |
| Stability | PANI-H_S62R- | 56.06 | 65.53 | 76.66 | 81.59 | 83.32 |
| Combo | L_F87Y | | | | | |
| Stability | PANI-L_F87Y- | 51.54 | 59.77/ | 76.62 | 81.67 | 83.33 |
| Combo | L_L96N | | 66.76 ** | | | |
| Stability | PANI-H_S62R- | 50.51 | 60.31/ | 76.42 | 81.64 | 83.32 |
| Combo | L_F87Y-L_L96N | | 66.33 ** | | | |
| Stability/ | PANI-H_S62R- | 59.69 | 67.89 | 76.66 | 81.42 | 83.25 |
| Affinity | L_F87Y-L_D92R | | | | | |
| Combo | | | | | | |
| Stability/ | PANI-H_S62R- | 60.62 | 68.82 | 76.84 | 81.51 | 83.27 |
| Affinity | L_F87Y-H_T98R | | | | | |
| Combo | | | | | | |
| Stability/ | PANI-H_S62R- | 60.55 | 69.43 | 76.65 | 81.14 | 83.10 |
| Affinity | L_F87Y- | | | | | |
| Combo | L_D92R-H_T98R | | | | | |
| Stability/ | PANI-L_F87Y- | 56.00 | 64.86 | 76.07 | 81.42 | 83.22 |
| Affinity | L_L96N-L_D92R | | | | | |
| Combo | | | | | | |
| Stability/ | PANI-L_F87Y- | 54.69 | 64.33 | 75.83 | 81.19 | 83.12 |
| Affinity | L_L96N-H_T98R | | | | | |
| Combo | | | | | | |
| Stability/ | PANI-H_S62R- | 55.72 | 65.18 | 75.95 | 81.25 | 83.13 |
| Affinity | L_F87Y-L_L96N- | | | | | |
| Combo | L_D92R | | | | | |
| Stability/ | PANI-H_S62R- | 55.67 | 64.86 | 75.66 | 81.01 | 83.04 |
| Affinity | L_F87Y-L_L96N- | | | | | |
| Combo | H_T98R | | | | | |
| Stability/ | PANI-H_S62R- | 57.17 | 66.55 | 75.97 | 81.18 | 83.15 |
| Affinity | L_F87Y-L_L96N- | | | | | |
| Combo | L_D92R-H_T98R | | | | | |
| Control | Original Pani-scFv | 53.25 | 64.09 | 76.17 | 81.44 | 83.28 |

*-H denotes mutation in the heavy chain; -L denotes mutation in the light chain; and PANI denotes the panitumumab framework.
** Additional peak was observed between peak 1 and peak 2.

PANI-H_S62R-L_F87Y-L_D92R (hereinafter “EGFR-scFv-3”) and PANI-H_S62R-L_F87Y (hereinafter “EGFR-scFv-2”) in Tables 6 and 7 demonstrated the best affinity and thermostability. The L:G100C and H: G44C mutations facilitate a disulfide bond to enhance the pairing of the VL and VH chains.

### Example 2. Surface Plasmon Resonance Analysis of Multispecific Binding Proteins

This example was designed to assess the binding affinity of certain panitumumab-derived EGFR-binding sites to EGFR. Four multispecific binding proteins, namely, EGFR-Protein-1, EGFR-Protein-2, EGFR-Protein-3, and EGFR-Protein-4, were constructed. These proteins contain an scFv having the amino acid sequences of EGFR-scFv-1, EGFR-scFv-2, EGFR-scFv-3, and EGFR-scFv-4, respectively, as disclosed in Table 1. Each of the multispecific binding proteins further contain a Fab fragment that binds an antigen unrelated to EGFR and an antibody Fc region. Kinetics and affinities of EGFR binding constructs for recombinant human and rhesus EGFR were measured by SPR using a Biacore 8K instrument. Samples were captured on the anti-hFc IgG chip and a range of concentrations of human or rhesus recombinant EGFR-His was injected over captured test articles. Experiments were performed at physiological temperature of 37° C. Data were analyzed using Biacore 8K Insight Evaluation software (GE Healthcare).

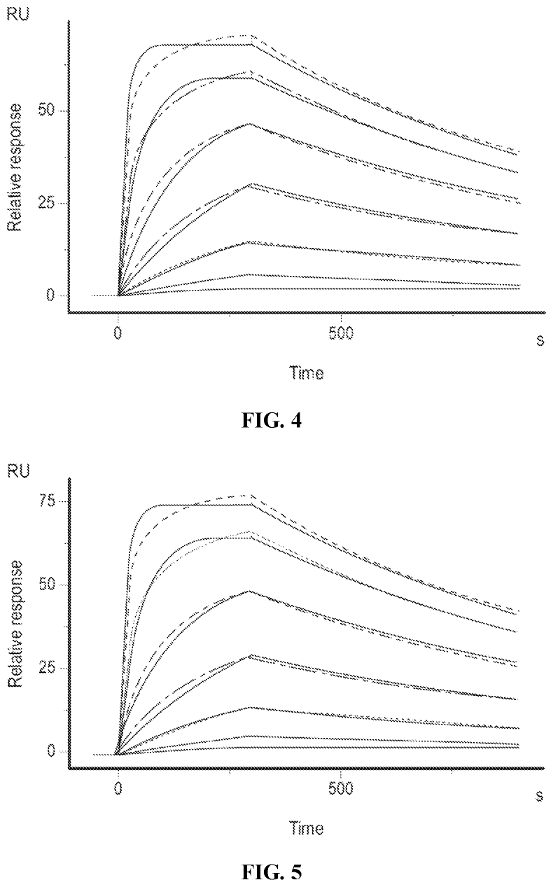

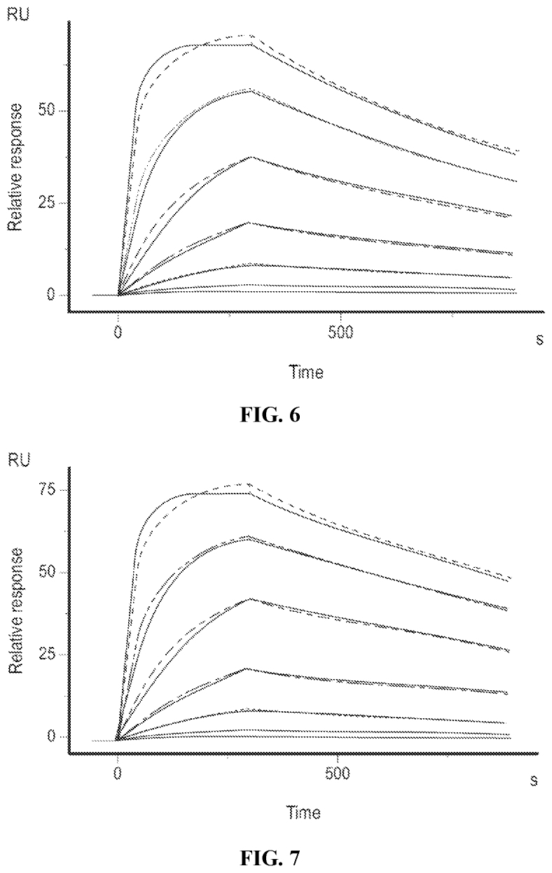

The binding sensorgrams from the SPR analysis are shown in FIGS. 4-7 and the parameters calculated from the analysis are shown in Table 8. FIG. 4 shows SPR analysis for EGFR-Protein-1, FIG. 5 shows SPR analysis for EGFR-Protein-2, FIG. 6 shows SPR analysis for EGFR-Protein-3, and FIG. 7 shows SPR analysis for EGFR-Protein-1. The results demonstrated that the mutations of interest did not have a substantial impact on the affinity of the multispecific binding proteins to EGFR.

TABLE 8
SPR Analysis: Human EGFR Binding (N = 4)

| | | Heavy | | | |
|---|---|---|---|---|---|
| | Light Chain | Chain | | | |
| | Mutations | Mutations | kₐ (M⁻¹ s⁻¹) | k_{d} (s⁻¹) | K_{D} (nM) |
| EGFR- | L: G100C | H: G44C | (5.92 ± 0.2)E+05 | (9.67 ± 0.05)E−04 | (1.64 ± 0.05)E−09 |
| Protein-1 | | | | | |
| EGFR- | L: G100C, | H: G44C, | (5.00 ± 0.2)E+05 | (9.88 ± 0.09)E−04 | (1.98 ± 0.07)E−09 |
| Protein-2 | F87Y | S62R | | | |
| EGFR- | L: G100C, | H: G44C, | (2.95 ± 0.1)E+05 | (9.30 ± 0.03)E−04 | (3.16 ± 0.1)E−09 |
| Protein-3 | F87Y, D92R | S62R | | | |
| EGFR- | L: G100C, | H: G44C | (3.16 ± 0.2)E+05 | (7.45 ± 0.08)E−04 | (2.36 ± 0.01)E−09 |
| Protein-4 | F87Y, D92R | | | | |

### Example 3. Differential Scanning Calorimetry (DSC) Analysis of Multispecific Binding Proteins

This example was designed to assess the thermostability of the multispecific binding protein constructs described in Example 2 using DSC analysis. Briefly, test articles were buffer-exchanged into the buffer of choice using Thermo Scientific Zeba Spin Desalting Columns. The eluate was then diluted to 0.5 mg/mL with the same buffer. 325 μL of the sample was loaded into a 96-well deepwell plate with the corresponding buffer blank and analyzed using a Microcal PEAQ-DSC instrument (Malvern Panalytical). The temperature was ramped from 20-25° C. to 100° C. at a rate of 60° C./hr. Buffer background was run in triplicate before the analytes. The most representative buffer blank was subtracted from each analyte scan prior to analysis. The data was fit using DSC analysis software with a non-two state model and Tₒₙₛₑₜ and Tₘs were reported.

EGFR-Protein-1 was used to obtain a melting curve as the baseline and identify the inflection points, especially the T-onset (the temperature under which the molecule starts melting) as well as Tm1, which is related to the stability of the scFv. Thereafter, EGFR-Protein-2, EGFR-Protein-3 and EGFR-Protein-4 were analyzed using the same method.

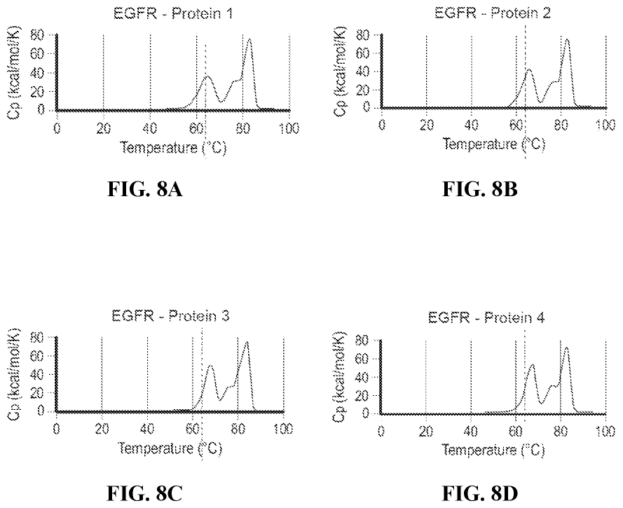

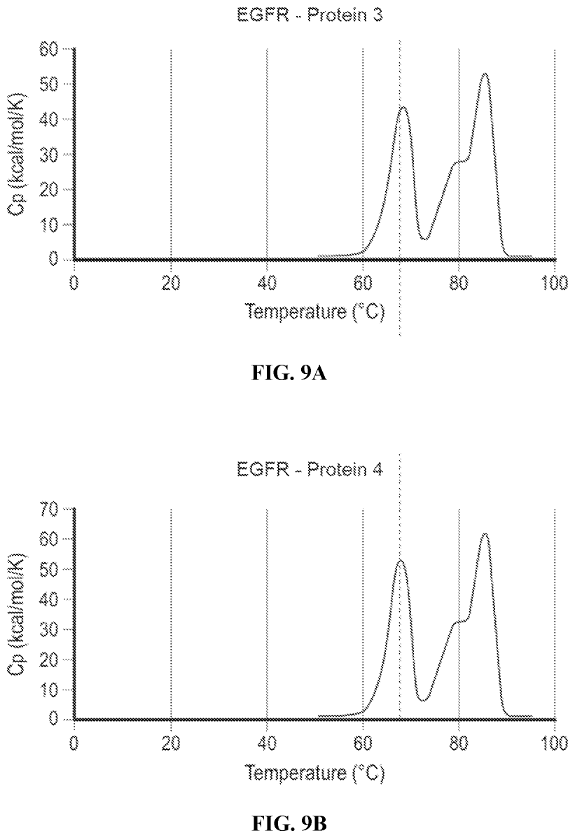

Table 9 and FIGS. 8A-8D show the DSC analysis results from testing in PBS, pH 7.4 buffer. FIG. 8A shows DSC analysis for EGFR-Protein-1 in PBS, pH 7.4 buffer. FIG. 8B shows DSC analysis for EGFR-Protein-2 in PBS, pH 7.4 buffer. FIG. 8C shows DSC analysis for EGFR-Protein-3 in PBS, pH 7.4 buffer. FIG. 8D shows DSC analysis for EGFR-Protein-4 in PBS, pH 7.4 buffer. Table 10 and FIGS. 9A and 9B show the DSC analysis results from testing in 20 mM histidine, 250 mM trehalose, 0.01% PS80, pH 6.0. FIG. 9A shows DSC analysis for EGFR-Protein-3 in 20 mM histidine, 250 mM trehalose, 0.01% PS80, pH 6.0 buffer. FIG. 9B shows DSC analysis for EGFR-Protein-3 in 20 mM histidine, 250 mM trehalose, 0.01% PS80, pH 6.0 buffer. The DSC analysis revealed that EGFR-Protein-3 (FIG. 8C) had improved thermostability relative to EGFR-Protein-2 (FIG. 8B), which in turn had improved thermostability relative to EGFR-Protein-1 (FIG. 8A). The results also showed similar thermostability of EGFR-Protein-3 (FIG. 9A) and EGFR-Protein-4 (FIG. 9B). However, EGFR-Protein-4 was more prone to degradation under accelerated thermostability studies (40° C. for 4 weeks, data not shown), which highlights the benefit of a S62R heavy chain mutation for stability.

TABLE 9
DSC analysis in PBS buffer, pH 7.4

| | Light | Heavy | | | | | |
|---|---|---|---|---|---|---|---|
| | Chain | Chain | Tonset, | Tm1, | Tm2, | Tm3, | Tm4, |
| Test article | Mutations | Mutations | (° C.) | (° C.) | (° C.) | (° C.) | (° C.) |
| EGFR- | G100C | G44C | 54.9 | 64.5 | 76.6 | 81.5 | 83.3 |
| Protein-1 | | | | | | | |
| EGFR- | G100C, | G44C, | 56.2 | 65.3 | 76.4 | 81.5 | 83.2 |
| Protein-2 | F87Y | S62R | | | | | |
| EGFR- | G100C, | G44C, | 59.8 | 67.5 | 76.3 | 81.3 | 83.1 |
| Protein-3 | F87Y, | S62 | | | | | |
| | D92R | | | | | | |
| EGFR- | G100C, | G44C | 58.6 | 66.9 | 76.1 | 81.3 | 83.2 |
| Protein-4 | F87Y, | | | | | | |
| | D92R | | | | | | |

TABLE 10
DSC analysis in 20 mM histidine, 250 mM trehalose, 0.01% PS80, pH 6.0

| | Light | Heavy | | | | | |
|---|---|---|---|---|---|---|---|
| | Chain | Chain | Tonset, | Tm1, | Tm2, | Tm3, | Tm4, |
| Test article | Mutations | Mutations | (° C.) | (° C.) | (° C.) | (° C.) | (° C.) |
| EGFR- | G100C, | G44C, | 59.0 | 67.8 | 79.1 | 84.2 | 86.3 |
| Protein-3 | F87Y, | S62 | | | | | |
| | D92R | | | | | | |
| EGFR- | G100C, | G44C | 58.7 | 67.4 | 79.1 | 84.3 | 86.3 |
| Protein-4 | F87Y, | | | | | | |
| | D92R | | | | | | |

### Example 4. Assessment of Multispecific Binding Protein Binding to EGFR Positive Cells

This Example was designed to assess the binding affinity of the EGFR-targeting multispecific binding proteins described in Example 2 to EGFR expressed on cell surface. The H2172 human cancer cell line, derived from non-small cell lung carcinoma, was used. Briefly, H2172 cells were incubated with EGFR-Protein-1, EGFR-Protein-2, EGFR-Protein-3 or panitumumab at 4° C. for 0.5 hours. After incubation, binding patterns of the multispecific binding proteins and panitumumab to EGFR⁺ cells were detected using a fluorophore-conjugated anti-human IgG secondary antibody. The levels of binding of the multispecific binding proteins to the cells were analyzed by flow cytometry.

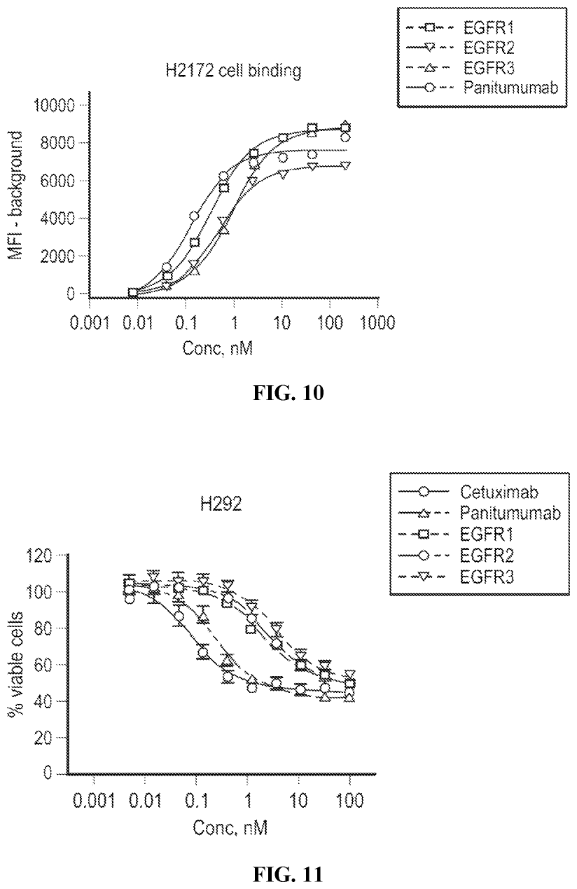

FIG. 10 shows binding to EGFR-positive cells after incubation with EGFR-Protein-1, EGFR-Protein-2, EGFR-Protein-3, or panitumumab. These EGFR-targeting multispecific binding proteins bound the cells with sub-nM concentrations and with similar or higher maximum binding than panitumumab.

### Example 5. EGFR Cell Proliferation Assay

This Example was designed to assess the impact of EGFR multispecific binding proteins on the proliferation of EGFR-expressing human cancer cell line H292. Briefly, the EGFR-multispecific binding proteins and anti-EGFR mAbs cetuximab and panitumumab were diluted and incubated with H292 cells for 72 hours. Following incubation, the cell proliferation was measured using Cell-titer Glo according to the manufacturer's instructions.

FIG. 11 shows the H292 cell proliferation in the presence of the EGFR-multispecific binding proteins or the anti-EGFR mAbs. The cells treated with the anti-EGFR mAbs showed less proliferation than those treated with the EGFR-multispecific binding proteins. This was attributed to the difference in the valency of the multispecific binding proteins and mAbs—the multispecific binding proteins are monovalent, while the mAbs are bivalent—and demonstrated the benefit of using an EGFR-targeting protein that has a lower valence, given the association of EGFR targeting with skin-related toxicity that has been observed in the mAbs.

### INCORPORATION BY REFERENCE

Unless stated to the contrary, the entire disclosure of each of the patent documents and scientific articles referred to herein is incorporated by reference for all purposes.

### EQUIVALENTS

An antigen-binding site described in application may be embodied in other specific forms without departing from the spirit or essential characteristics thereof. The foregoing embodiments are therefore to be considered in all respects illustrative rather than limiting on an antigen-binding site described herein. Scope of the present application is thus indicated by the appended claims rather than by the foregoing description, and all changes that come within the meaning and the range of equivalency of the claims are intended to be embraced therein.

What is claimed is:

1. An antigen-binding site that binds EGFR, comprising:
(i) a heavy chain variable domain (VH) comprising complementarity-determining region 1 (CDR1), complementarity-determining region 2 (CDR2), and complementarity-determining region 3 (CDR3) sequences of SEQ ID NOs: 2, 23, and 4, respectively; and a light chain variable domain (VL) comprising CDR1, CDR2, and CDR3 sequences of SEQ ID NOs: 6, 7, and 17, respectively; or
(ii) a VH comprising CDR1, CDR2, and CDR3 sequences of SEQ ID NOs: 2, 12, and 4, respectively; and a VL comprising CDR1, CDR2, and CDR3 sequences of SEQ ID NOs: 6, 7, and 8, respectively.

2. The antigen-binding site of claim 1, wherein the VH comprises CDR1, CDR2, and CDR3 sequences of SEQ ID NOs: 2, 23, and 4, respectively; and the VL comprises CDR1, CDR2, and CDR3 sequences of SEQ ID NOs: 6, 7, and 17, respectively.

3. The antigen-binding site of claim 2, where the VH comprises CDR1, CDR2, and CDR3 sequences of SEQ ID NOs: 2, 12, and 4, respectively; and the VL comprises CDR1, CDR2, and CDR3 sequences of SEQ ID NOs: 6, 7, and 17, respectively.

4. The antigen-binding site of claim 2, wherein the VH comprises CDR1, CDR2, and CDR3 sequences of SEQ ID NOs: 2, 3, and 4, respectively; and the VL comprises CDR1, CDR2, and CDR3 sequences of SEQ ID NOs: 6, 7, and 17, respectively.

5. The antigen-binding site of claim 1, wherein the VH comprises CDR1, CDR2, and CDR3 sequences of SEQ ID NOs: 2, 12, and 4, respectively; and the VL comprises CDR1, CDR2, and CDR3 sequences of SEQ ID NOs: 6, 7, and 8, respectively.

6. An antigen-binding site comprising: a VH comprising an amino acid sequence at least 90% identical to SEQ ID NO:1 and a VL comprising an amino acid sequence at least 90% identical to SEQ ID NO:5, wherein the VH comprises an S62R substitution relative to SEQ ID NO:1 and/or the VL comprises a D92R substitution and/or an F87Y substitution relative to SEQ ID NO:5, numbered under the Chothia numbering scheme.

7. The antigen-binding site of claim 6, wherein the VH comprises an S62R substitution relative to SEQ ID NO:1, numbered under the Chothia numbering scheme.

8. The antigen-binding site of claim 6, wherein the VL comprises a D92R substitution relative to SEQ ID NO:5, numbered under the Chothia numbering scheme.

9. The antigen-binding site of claim 6, wherein the VH comprises an S62R substitution relative to SEQ ID NO:1 and the VL comprises a D92R substitution relative to SEQ ID NO:5, numbered under the Chothia numbering scheme.

10. The antigen-binding site of any one of claims 1-9, wherein the VL comprises an F87Y substitution relative to SEQ ID NO:5, numbered under the Chothia numbering scheme.

11. The antigen-binding site of any one of claims 1-3, and 6-10, wherein the VH comprises an amino acid sequence at least 90% identical to SEQ ID NO:11 and the VL comprises an amino acid sequence at least 90% identical to SEQ ID NO: 16.

12. The antigen-binding site of any one of claims 1-3, and 6-11, wherein the VH comprises the amino acid sequence of SEQ ID NO:11 and the VL comprises the amino acid sequence of SEQ ID NO:16, or the VH comprises the amino acid sequence of SEQ ID NO:32 and the VL comprises the amino acid sequence of SEQ ID NO:33.

13. The antigen-binding site of any one of claims 1, 2, 4, 6, 8, and 10, wherein the VH comprises an amino acid sequence at least 90% identical to SEQ ID NO:1 and the VL comprises an amino acid sequence at least 90% identical to SEQ ID NO:16.

14. The antigen-binding site of any one of claims 1, 2, 4, 6, 8, 10, and 13, wherein the VH comprises the amino acid sequence of SEQ ID NO:1 and the VL comprises the amino acid sequence of SEQ ID NO:16.

15. The antigen-binding site of claim 1, 5-7, and 10, wherein the VH comprises an amino acid sequence at least 90% identical to SEQ ID NO:11 and the VL comprises an amino acid sequence at least 90% identical to SEQ ID NO: 13.

16. The antigen-binding site of claim 1, 5-7, 10, and 15, wherein the VH comprises the amino acid sequence of SEQ ID NO:11 and the VL comprises the amino acid sequence of SEQ ID NO: 13.

17. The antigen-binding site of any one of claims 1-16, wherein the antigen-binding site is present as a single-chain fragment variable (scFv), and wherein the scFv comprises a sequence selected from SEQ ID NOs: 14, 18, 20, and 24.

18. The antigen-binding site of any one of claims 1-17, wherein the antigen-binding site binds human EGFR with a dissociation constant (K_{D}) smaller than or equal to 5 nM, as measured by surface plasmon resonance (SPR).

19. The antigen-binding site of any one of claims 1-18, wherein the antigen-binding site binds rhesus macaque EGFR with a dissociation constant (K_{D}) smaller than or equal to 6 nM, as measured by surface plasmon resonance (SPR).

20. A protein comprising the antigen-binding site of any one of the claims 1-19.

21. The protein of claim 20, further comprising an antibody heavy chain constant region.

22. The protein of claim 21, wherein the antibody heavy chain constant region is a human IgG heavy chain constant region.

23. The protein of claim 22, wherein the antibody heavy chain constant region is a human IgG1 heavy chain constant region.

24. The protein of claim 22 or 23, wherein each polypeptide chain of the antibody heavy chain constant region comprises an amino acid sequence at least 90% identical to SEQ ID NO:26.

25. The protein of any one of claims 22-24, wherein at least one polypeptide chain of the antibody heavy chain constant region comprises one or more mutations, relative to SEQ ID NO:26, at one or more positions selected from Q347, Y349, L351, S354, E356, E357, K360, Q362, S364, T366, L368, K370, N390, K392, T394, D399, S400, D401, F405, Y407, K409, T411, and K439, numbered according to the EU numbering system.

26. The protein of any one of claims 22-25, wherein at least one polypeptide chain of the antibody heavy chain constant region comprises one or more mutations, relative to SEQ ID NO:26, selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E, numbered according to the EU numbering system.

27. The protein of any one of claims 22-26, wherein one polypeptide chain of the antibody heavy chain constant region comprises one or more mutations, relative to SEQ ID NO:26, at one or more positions selected from Q347, Y349, L351, S354, E356, E357, K360, Q362, S364, T366, L368, K370, K392, T394, D399, 5400, D401, F405, Y407, K409, T411 and K439; and the other polypeptide chain of the antibody heavy chain constant region comprises one or more mutations, relative to SEQ ID NO:26, at one or more positions selected from Q347, Y349, L351, S354, E356, E357, S364, T366, L368, K370, N390, K392, T394, D399, D401, F405, Y407, K409, T411, and K439, numbered according to the EU numbering system.

28. The protein of claim 27, wherein one polypeptide chain of the antibody heavy chain constant region comprises K360E and K409W substitutions relative to SEQ ID NO:26; and the other polypeptide chain of the antibody heavy chain constant region comprises Q347R, D399V and F405T substitutions relative to SEQ ID NO:26, numbered according to the EU numbering system.

29. The protein of claim 27 or 28, wherein one polypeptide chain of the antibody heavy chain constant region comprises a Y349C substitution relative to SEQ ID NO:26; and the other polypeptide chain of the antibody heavy chain constant region comprises an S354C substitution relative to SEQ ID NO:26, numbered according to the EU numbering system.

30. An antibody-drug conjugate comprising the protein of any one of claims 20-29 and a drug moiety.

31. The antibody-drug conjugate of claim 30, wherein the drug moiety is selected from the group consisting of auristatin, N-acetyl-7 calicheamicin, maytansinoid, pyrrolobenzodiazepine, and SN-38.

32. An immunocytokine comprising the antigen-binding site of any one of claims 1-19 and a cytokine.

33. The immunocytokine of claim 32, wherein the cytokine is selected from the group consisting of IL-2, IL-4, IL-10, IL-12, IL-15, TNF, and IFNα.

34. A bispecific T-cell engager comprising the antigen-binding site of any one of claims 1-19 and an antigen-binding site that binds CD3.

35. A chimeric antigen receptor (CAR) comprising:
(a) the antigen-binding site of any one of claims 1-19;
(b) a transmembrane domain; and
(c) an intracellular signaling domain.

36. The CAR of claim 35, wherein the transmembrane domain is selected from the transmembrane regions of the alpha, beta or zeta chain of the T-cell receptor, CD28, CD3 epsilon, CD45, CD4, CD5, CD8, CD9, CD16, CD22, EGFR, CD37, CD64, CD80, CD86, CD134, CD137, CD152, and CD154.

37. The CAR of claim 35 or 36, wherein the intracellular signaling domain comprises a primary signaling domain comprising a functional signaling domain of CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12.

38. The CAR of any one of claims 35-37, wherein the intracellular signaling domain further comprises a costimulatory signaling domain comprising a functional signaling domain of a costimulatory receptor.

39. The CAR of claim 38, wherein the costimulatory receptor is selected from the group consisting of OX40, CD27, CD28, CD30, CD40, PD-1, CD2, CD7, CD258, NKG2C, B7-H3, a ligand that binds to CD83, ICAM-1, LFA-1 (CD11a/CD18), ICOS and 4-1BB (CD137), or any combination thereof.

40. An isolated nucleic acid encoding the CAR of any one of claims 35-39.

41. An expression vector comprising the isolated nucleic acid of claim 40.

42. An immune effector cell comprising the nucleic acid of claim 40 or the expression vector of claim 41.

43. An immune effector cell expressing the CAR of any one of claims 35-39.

44. The immune effector cell of claim 42 or 43, wherein the immune effector cell is a T cell.

45. The immune effector cell of claim 44, wherein the T cell is a CD8⁺ T cell, a CD4⁺ T cell, a γδ T cell, or an NKT cell.

46. The immune effector cell of claim 42 or 43, wherein the immune effector cell is an NK cell.

47. A pharmaceutical composition comprising the protein of any one of claims 20-29, the antibody-drug conjugate of claim 30 or 31, the immunocytokine of claim 32 or 33, the bispecific T-cell engager of claim 34, or the immune effector cell of any one of claims 42-46; and a pharmaceutically acceptable carrier.

48. A method of treating cancer, the method comprising administering to a subject in need thereof an effective amount of the protein of any one of claims 20-29, the antibody-drug conjugate of claim 30 or 31, the immunocytokine of claim 32 or 33, the bispecific T-cell engager of claim 34, or the immune effector cell of any one of claims 42-46, or the pharmaceutical composition of claim 47.

49. The method of claim 48, wherein the cancer is a solid tumor.

50. The method of claim 48 or 49, wherein the cancer is lung cancer, breast cancer, kidney cancer, colorectal cancer, gastric cancer, brain cancer, glioma, bladder cancer, head and neck cancer, bladder cancer, pancreatic cancer, and liver cancer, cervical cancer, ovarian cancer or prostate cancer.

51. The method of any one of claims 48-50, wherein the cancer expresses EGFR.

52. The antigen-binding site of any one of claims 1-19, the protein of any one of claims 20-29, the antibody-drug conjugate of claim 30 or claim 31, the immunocytokine of claim 32 or claim 33, or the bispecific T cell engager of claim 34, wherein the antigen-binding site, protein, antibody-drug conjugate, immunocytokine, or bispecific T cell engager is a purified antigen-binding site, protein, antibody-drug conjugate, immunocytokine, or bispecific T cell engager.

53. The antigen-binding site, protein, antibody-drug conjugate, immunocytokine, or bispecific T cell engager of claim 52, wherein the antigen-binding site, protein, antibody-drug conjugate, immunocytokine, or bispecific T cell engager is purified by a method selected from the group consisting of: centrifugation, depth filtration, cell lysis, homogenization, freeze-thawing, affinity purification, gel filtration, ion exchange chromatography, hydrophobic interaction exchange chromatography, and mixed-mode chromatography.


SEQUENCE LISTING (33 sequences)

SEQ ID NO: 1 - protein, 119 aa, Artificial Sequence [sequence omitted]

SEQ ID NO: 2 - protein, 12 aa, Artificial Sequence
  Description of Artificial Sequence Synthetic peptide
GGSVSSGDYYWT

SEQ ID NO: 3 - protein, 16 aa, Artificial Sequence
  Description of Artificial Sequence Synthetic peptide
HIYYSGNTNYNPSLKS

SEQ ID NO: 4 - protein, 9 aa, Artificial Sequence
  Description of Artificial Sequence Synthetic peptide
DRVTGAFDI

SEQ ID NO: 5 - protein, 107 aa, Artificial Sequence [sequence omitted]

SEQ ID NO: 6 - protein, 11 aa, Artificial Sequence
  Description of Artificial Sequence Synthetic peptide
QASQDISNYLN

SEQ ID NO: 7 - protein, 11 aa, Artificial Sequence
  Description of Artificial Sequence Synthetic peptide
LLIYDASNLET

SEQ ID NO: 8 - protein, 9 aa, Artificial Sequence
  Description of Artificial Sequence Synthetic peptide
QHFDHLPLA

SEQ ID NO: 9 - protein, 246 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSS

SEQ ID NO: 10 - protein, 246 aa, Artificial Sequence
  Description of Artificial Sequence Synthetic polypeptide
QVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGCGTKVEIK

SEQ ID NO: 11 - protein, 119 aa, Artificial Sequence
  Description of Artificial Sequence Synthetic polypeptide
QVQLQESGPGLVKPSETLSLTCTVSGGSVSSGDYYWTWIRQSPGKGLEWIGHIYYSGNTNYNPRLKSRLTISIDTSKTQFSLKLSSVTAADTAIYYCVRDRVTGAFDIWGQGTMVTVSS

SEQ ID NO: 12 - protein, 16 aa, Artificial Sequence
  Description of Artificial Sequence Synthetic peptide
HIYYSGNTNYNPRLKS

SEQ ID NO: 13 - protein, 107 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQHFDHLPLAFGGGTKVEIK

SEQ ID NO: 14 - protein, 246 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQHFDHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSS

SEQ ID NO: 15 - protein, 246 aa, Artificial Sequence
  Description of Artificial Sequence Synthetic polypeptide
QVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQHFDHLPLAFGCGTKVEIK

SEQ ID NO: 16 - protein, 107 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGGGTKVEIK

SEQ ID NO: 17 - protein, 9 aa, Artificial Sequence
  Description of Artificial Sequence Synthetic peptide
QHFRHLPLA

SEQ ID NO: 18 - protein, 246 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSS

SEQ ID NO: 19 - protein, 246 aa, Artificial Sequence
  Description of Artificial Sequence Synthetic polypeptide
QVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIK

SEQ ID NO: 20 - protein, 246 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSS

SEQ ID NO: 21 - protein, 246 aa, Artificial Sequence
  Description of Artificial Sequence Synthetic polypeptide
QVQLQESGPGLVKPSETLSLTCTVSGGSVSSGDYYWTWIRQSPGKCLEWIGHIYYSGNTNYNPSLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIK

SEQ ID NO: 22 - protein, 119 aa, Artificial Sequence
  Description of Artificial Sequence Synthetic polypeptide
  MOD_RES (64)..(64): Arg or Ser
QVQLQESGPGLVKPSETLSLTCTVSGGSVSSGDYYWTWIRQSPGKGLEWIGHIYYSGNTNYNPXLKSRLTISIDTSKTQFSLKLSSVTAADTAIYYCVRDRVTGAFDIWGQGTMVTVSS

SEQ ID NO: 23 - protein, 16 aa, Artificial Sequence
  Description of Artificial Sequence Synthetic peptide
  MOD_RES (13)..(13): Arg or Ser
HIYYSGNTNYNPXLKS

SEQ ID NO: 24 - protein, 246 aa, Artificial Sequence
  Description of Artificial Sequence Synthetic polypeptide
  MOD_RES (191)..(191): Arg or Ser
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIKGGGGSGGGGSGGGGSGGGGSQVQLQESGPGLVKPSETLSLTCTVSGGSVSSGDYYWTWIRQSPGKCLEWIGHIYYSGNTNYNPXLKSRLTISIDTSKTQFSLKLSSVTAADTAIYYCVRDRVTGAFDIWGQGTMVTVSS

SEQ ID NO: 25 - protein, 246 aa, Artificial Sequence
  Description of Artificial Sequence Synthetic polypeptide
  MOD_RES (64)..(64): Arg or Ser
QVQLQESGPGLVKPSETLSLTCTVSGGSVSSGDYYWTWIRQSPGKCLEWIGHIYYSGNTNYNPXLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIK

SEQ ID NO: 26 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 1210 aa, Homo sapiens [sequence omitted]

SEQ ID NO: 28 - protein, 405 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGLS

SEQ ID NO: 29 - protein, 705 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 30 - protein, 628 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGS

SEQ ID NO: 31 - protein, 20 aa, Artificial Sequence
  Description of Artificial Sequence Synthetic peptide
GGGGSGGGGSGGGGSGGGGS

SEQ ID NO: 32 - protein, 119 aa, Artificial Sequence
  Description of Artificial Sequence Synthetic polypeptide
QVQLQESGPGLVKPSETLSLTCTVSGGSVSSGDYYWTWIRQSPGKCLEWIGHIYYSGNTNYNPRLKSRLTISIDTSKTQFSLKLSSVTAADTAIYYCVRDRVTGAFDIWGQGTMVTVSS

SEQ ID NO: 33 - protein, 107 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQHFRHLPLAFGCGTKVEIK